SEQUENCE LISTING (88 sequences)

SEQ ID NO: 1 - protein, 116 aa, Artificial sequence
  An heavy chain variable region of an anti-VEGF antibody or anti-VEGFR antibody in fusion protein
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQG

SEQ ID NO: 2 - protein, 101 aa, Artificial sequence
  An light chain variable region of an anti-VEGF antibody or anti-VEGFR antibody in fusion protein
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQG

SEQ ID NO: 3 - protein, 116 aa, Artificial sequence
  An heavy chain variable region of an anti-VEGF antibody or anti-VEGFR antibody in fusion protein
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQG

SEQ ID NO: 4 - protein, 101 aa, Artificial sequence
  An light chain variable region of an anti-VEGF antibody or anti-VEGFR antibody in fusion protein
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQG

SEQ ID NO: 5 - protein, 109 aa, Artificial sequence
  An heavy chain variable region of an anti-VEGF antibody or anti-VEGFR antibody in fusion protein
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQG

SEQ ID NO: 6 - protein, 101 aa, Artificial sequence
  An light chain variable region of an anti-VEGF antibody or anti-VEGFR antibody in fusion protein
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGG

SEQ ID NO: 7 - protein, 205 aa, Artificial sequence
  VEGFR1-D2/VEGFR2-D3
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 8 - protein, 303 aa, Artificial sequence
  VEGFR1-D2/VEGFR2-D3-D4
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPGPGD

SEQ ID NO: 9 - protein, 100 aa, Artificial sequence
  VEGFR1-D2
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNT

SEQ ID NO: 10 - protein, 227 aa, Artificial sequence
  An amino acid sequence of IgG1 Fc in the fusion protein
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 232 aa, Artificial sequence
  An amino acid sequence of IgG2 Fc in fusion protein
DKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 233 aa, Artificial sequence
  An amino acid sequence of IgG4 Fc in the fusion protein
DKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 112 aa, Artificial sequence
  CD80-IgV
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPS

SEQ ID NO: 14 - protein, 208 aa, Artificial sequence [sequence omitted]

SEQ ID NO: 15 - protein, 113 aa, Artificial sequence
  A light chain constant region sequence of an anti-VEGF antibody or anti-VEGFR antibody in the fusion protein, type kappa
TKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 112 aa, Artificial sequence
  A light chain constant region sequence of an anti-VEGF antibody or anti-VEGFR antibody in the fusion protein, lambda type
TKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 17 - protein, 337 aa, Artificial sequence
  A heavy chain constant region sequence of an antibody in the fusion protein, IgG1 type
TLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 333 aa, Artificial sequence
  A heavy chain constant region sequence of an antibody in the fusion protein, IgG2 type
TTVTVSTASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - protein, 334 aa, Artificial sequence
  A heavy chain constant region sequence of an antibody in the fusion protein, IgG4 type
TLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 16 aa, Artificial sequence
  A peptide linker in the fusion protein
AKTTPKLEEGEFSEAR

SEQ ID NO: 21 - protein, 17 aa, Artificial sequence
  A peptide linker in the fusion protein
AKTTPKLEEGEFSEARV

SEQ ID NO: 22 - protein, 9 aa, Artificial sequence
  A peptide linker in the fusion protein
AKTTPKLGG

SEQ ID NO: 23 - protein, 10 aa, Artificial sequence
  A peptide linker in the fusion protein
SAKTTPKLGG

SEQ ID NO: 24 - protein, 6 aa, Artificial sequence
  A peptide linker in the fusion protein
SAKTTP

SEQ ID NO: 25 - protein, 6 aa, Artificial sequence
  A peptide linker in the fusion protein
RADAAP

SEQ ID NO: 26 - protein, 9 aa, Artificial sequence
  A peptide linker in the fusion protein
RADAAPTVS

SEQ ID NO: 27 - protein, 12 aa, Artificial sequence
  A peptide linker in the fusion protein
RADAAAAGGPGS

SEQ ID NO: 28 - protein, 7 aa, Artificial sequence
  A peptide linker in the fusion protein
RADAAAA

SEQ ID NO: 29 - protein, 18 aa, Artificial sequence
  A peptide linker in the fusion protein
SAKTTPKLEEGEFSEARV

SEQ ID NO: 30 - protein, 5 aa, Artificial sequence
  A peptide linker in the fusion protein
ADAAP

SEQ ID NO: 31 - protein, 11 aa, Artificial sequence
  A peptide linker in the fusion protein
DAAPTVSIFPP

SEQ ID NO: 32 - protein, 5 aa, Artificial sequence
  A peptide linker in the fusion protein
TVAAP

SEQ ID NO: 33 - protein, 12 aa, Artificial sequence
  A peptide linker in the fusion protein
TVAAPSVFIFPP

SEQ ID NO: 34 - protein, 6 aa, Artificial sequence
  A peptide linker in the fusion protein
QPKAAP

SEQ ID NO: 35 - protein, 13 aa, Artificial sequence
  A peptide linker in the fusion protein
QPKAAPSVTLFPP

SEQ ID NO: 36 - protein, 6 aa, Artificial sequence
  A peptide linker in the fusion protein
AKTTPP

SEQ ID NO: 37 - protein, 13 aa, Artificial sequence
  A peptide linker in the fusion protein
AKTTPPSVTPLAP

SEQ ID NO: 38 - protein, 6 aa, Artificial sequence
  A peptide linker in the fusion protein
AKTTAP

SEQ ID NO: 39 - protein, 13 aa, Artificial sequence
  A peptide linker in the fusion protein
AKTTAPSVYPLAP

SEQ ID NO: 40 - protein, 6 aa, Artificial sequence
  A peptide linker in the fusion protein
ASTKGP

SEQ ID NO: 41 - protein, 13 aa, Artificial sequence
  A peptide linker in the fusion protein
ASTKGPSVFPLAP

SEQ ID NO: 42 - protein, 15 aa, Artificial sequence
  A peptide linker in the fusion protein
GGGGSGGGGSGGGGS

SEQ ID NO: 43 - protein, 15 aa, Artificial sequence
  A peptide linker in the fusion protein
GENKVEYAPALMALS

SEQ ID NO: 44 - protein, 15 aa, Artificial sequence
  A peptide linker in the fusion protein
GPAKELTPLKEAKVS

SEQ ID NO: 45 - protein, 15 aa, Artificial sequence
  A peptide linker in the fusion protein
GHEAAAVMQVQYPAS

SEQ ID NO: 46 - protein, 16 aa, Artificial sequence
  A peptide linker in the fusion protein
GGGGSGGGGSGGGGSA

SEQ ID NO: 47 - protein, 106 aa, Artificial sequence
  A heavy chain variable region sequence of an anti-PD-1 antibody in combination therapy
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQG

SEQ ID NO: 48 - protein, 101 aa, Artificial sequence
  A light chain variable region sequence of an anti-PD-1 antibody in combination therapy
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQG

SEQ ID NO: 49 - protein, 113 aa, Artificial sequence
  A heavy chain variable region sequence of an anti-PD-1 antibody in combination therapy
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQG

SEQ ID NO: 50 - protein, 105 aa, Artificial sequence
  A light chain variable region sequence of an anti-PD-1 antibody in combination therapy
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGG

SEQ ID NO: 51 - protein, 110 aa, Artificial sequence
  A heavy chain variable region sequence of an anti-PD-1 antibody in combination therapy
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQG

SEQ ID NO: 52 - protein, 100 aa, Artificial sequence
  A light chain variable region sequence of an anti-PD-1 antibody in combination therapy
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGG

SEQ ID NO: 53 - protein, 109 aa, Artificial sequence
  A heavy chain variable region sequence of an anti-PD-1 antibody in combination therapy
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQG

SEQ ID NO: 54 - protein, 101 aa, Artificial sequence
  A light chain variable region sequence of an anti-PD-1 antibody in combination therapy
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGG

SEQ ID NO: 55 - protein, 114 aa, Artificial sequence
  A heavy chain variable region sequence of an anti-PD-1 antibody in combination therapy
QVTLKESGPALVKPTQTLTLTCTFSGFSLSTSGTCVSWIRQPPGKALEWLATICWEDSKGYNPSLKSRLTISKDTSKNQAVLTMTNMDPVDTATYYCARREDSGYFWFPYWGQG

SEQ ID NO: 56 - protein, 100 aa, Artificial sequence
  A light chain variable region sequence of an anti-PD-1 antibody in combination therapy
NIQMTQSPSSLSASVGDRVTITCKAGQNVNNYLAWYQQKPGKAPKVLIFNANSLQTGVPSRFSGSGSGTDFTLTISSLQPEDFATYYCQQYNSWTTFGGG

SEQ ID NO: 57 - protein, 111 aa, Artificial sequence
  A heavy chain variable region sequence of an anti-PD-1 antibody in combination therapy
QVQLQESGPGLVKPSETLSLTCTVSGFSLTSYGVHWIRQPPGKGLEWLGVIWAGGSTNYNPSLKSRLTISKDNSKSQVSLKMSSVTAADTAVYYCARAYGNYWYIDVWGQG

SEQ ID NO: 58 - protein, 101 aa, Artificial sequence
  A light chain variable region sequence of an anti-PD-1 antibody in combination therapy
DIVMTQSPDSLAVSLGERATINCKASQSVSNDVAWYQQKPGQPPKLLINYAFHRFTGVPDRFSGSGYGTDFTLTISSLQAEDVAVYYCHQAYSSPYTFGGG

SEQ ID NO: 59 - protein, 105 aa, Artificial sequence
  A heavy chain variable region sequence of an anti-PD-1 antibody in combination therapy
VQLVESGGGVVQPGRSLRLDCKASGITFSNYGMHWVRQAPGKGLEWVAVIWYDSSRKYYADSVKGRFTISRDNSKNTLFLQMNSLRAEDTAVYYCATNNDYWGQG

SEQ ID NO: 60 - protein, 101 aa, Artificial sequence
  A light chain variable region sequence of an anti-PD-1 antibody in combination therapy
DIQMTQSPSSLSASVGDRVTITCRASQSVSNYLDWYQQKPGKAPKLLIYDASTRATGVPSRFSGSGSGTDFTLTISSLQPEDFATYYCQQNMQLPLTFGQG

SEQ ID NO: 61 - protein, 113 aa, Artificial sequence
  A heavy chain variable region sequence of an anti-PD-1 antibody in combination therapy
DLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGRGYSYGIDAFDIWGQG

SEQ ID NO: 62 - protein, 103 aa, Artificial sequence
  A light chain variable region sequence of an anti-PD-1 antibody in combination therapy
LSYVLTQPPSVSVSPGQTARITCSGDALPKQYAYWYQQKPGQAPVLVIYKDSERPSGIPERFSGSSSGTTVTLTISGVQAEDEADYYCQSADSSGTYVVFGGG

SEQ ID NO: 63 - protein, 118 aa, Artificial sequence
  A heavy chain variable region sequence of an anti-PD-1 antibody in combination therapy
QGQLVQSGAEVKKPGASVKVSCKASGYTFTDYEMHWVRQAPGQGLEWMGVIESETGGTAYNQKFQGRVTLTADKSSSTAYMELSSLRSEDTAVYYCTREGITTVATTYYWYFDVWGQG

SEQ ID NO: 64 - protein, 106 aa, Artificial sequence
  A light chain variable region sequence of an anti-PD-1 antibody in combination therapy
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQG

SEQ ID NO: 65 - protein, 111 aa, Artificial sequence
  A heavy chain variable region sequence of an anti-PD-1 antibody in combination therapy
EVQLVESGGGLVQPGGSLRLSCAASGFTFSSYGMSWVRQAPGKGLEWVATISGGGSDTYYADSVKGRFTISRDNSKNTLYLQMNSLRAEDTAVYYCARQLNYAWFAYWGQG

SEQ ID NO: 66 - protein, 105 aa, Artificial sequence
  A light chain variable region sequence of an anti-PD-1 antibody in combination therapy
DIVLTQSPASLAVSPGQRATITCRASESVDNYGISFMNWYQQKPGQPPKLLIYTSSNKDTGVPARFSGSGSGTDFTLTINPMEAEDTAVYYCQQSKEVPWTFGGG

SEQ ID NO: 67 - protein, 113 aa, Artificial sequence
  A heavy chain variable region sequence of an anti-PD-1 antibody in combination therapy
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQG

SEQ ID NO: 68 - protein, 101 aa, Artificial sequence
  A light chain variable region sequence of an anti-PD-1 antibody in combination therapy
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGG

SEQ ID NO: 69 - protein, 116 aa, Artificial sequence
  A heavy chain variable region sequence of an anti-PD-1 antibody in combination therapy
QLQLQESGPGLVKPSETLTLTCTVSADSISSTTYYWVWIRQPPGKGLEWIGSISYSGSTYYNPSLKSRVTVSVDTSKNQFSLKLNSVAATDTALYYCARHLGYNGRYLPFDYWGQG

SEQ ID NO: 70 - protein, 104 aa, Artificial sequence
  A light chain variable region sequence of an anti-PD-1 antibody in combination therapy
QSALTQPASVSGSPGQSITISCTGTSSDVGFYNYVSWYQQHPGKAPELMIYDVSNRPSGVSDRFSGSKSGNTASLTISGLQAEDEADYYCSSYTNISTWVFGGG

SEQ ID NO: 71 - protein, 106 aa, Artificial sequence
  A heavy chain variable region sequence of an anti-PD-1 antibody in combination therapy
QVQLVESGGGVVQPGRSLRLTCKASGLTFSSSGMHWVRQAPGKGLEWVAVIWYDGSKRYYADSVKGRFTISRDNSKNTLFLQMNSLRAEDTAVYYCATNNDYWGQG

SEQ ID NO: 72 - protein, 101 aa, Artificial sequence
  A light chain variable region sequence of an anti-PD-1 antibody in combination therapy
EIVLTQSPATLSLSPGERATLSCRASQSVSSYLAWYQQKPGQAPRLLIYTASNRATGIPARFSGSGSGTDFTLTISSLEPEDFAVYYCQQYSNWPRTFGQG

SEQ ID NO: 73 - DNA, 723 bp, Artificial sequence
  A nucleotide sequence of the light chain (BY18.1L) of the anti-PD1 antibody BY18.1
ctcgaggccaccatggagaccgacacactcctcctgtgggtgctgctgctgtgggtgcctggctccactggcgagattgtgctgacacagtcccccgctactctgagcctgagccctggcgagagggctacactgtcttgcagagcttctcagtccgtgtcttcttacctcgcttggtatcagcagaagcccggccaggctccaagactgctgatctatgacgcttctaaccgcgctacaggcattcctgctaggttcagcggcagcggctctggcaccgacttcacactcacaattagctctcttgaacctgaggacttcgccgtgtactactgccagcagtctagcaactggcctagaacattcggccagggcactaaggtggagattaagagaaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaagaattc

SEQ ID NO: 74 - protein, 234 aa, Artificial sequence
  An amino acid sequence of light chain (BY18.1L) of anti-PD1 antibody BY18.1
METDTLLLWVLLLWVPGSTGEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 1401 bp, Artificial sequence
  A nucleotide sequence of heavy chain (BY18.1H) of anti-PD1 antibody BY18.1
tctagagccaccatggagaccgacaccctgctgctgtgggtgctgctcctgtgggtgcctggctccacaggccaggtgcagctcgtggagtccggcggcggcgtggtgcagcccggcagatccctcagactggactgcaaggcatccggcattacattctctaactctggaatgcactgggtgagacaggctcctggcaagggcctggaatgggtggccgtgatttggtacgacggctctaagagatactacgctgactccgtgaagggccggttcacaattagcagagacaactccaagaacactctgttcctccagatgaacagcctgagagccgaggacaccgctgtgtactactgcgccaccaacgacgactactggggccagggcaccctcgtgacagtgtcttccgcctccaccaagggcccttccgtgttccctctggccccttgctcccgctccacctccgagtccaccgccgccctgggctgcctggtgaaggactacttccctgagcctgtgaccgtgtcctggaactccggcgccctgacctccggcgtgcacaccttccctgccgtgctgcagtcctccggcctgtactccctgtcctccgtggtgaccgtgccttcctcctccctgggcaccaagacctacacctgcaacgtggaccacaagccttccaacaccaaggtggacaagcgcgtggagtccaagtacggccctccttgccctccttgccctgcccctgagttcctgggcggcccttccgtgttcctgttccctcctaagcctaaggacaccctgatgatctcccgcacccctgaggtgacctgcgtggtggtggacgtgtcccaggaggaccctgaggtgcagttcaactggtacgtggacggcgtggaggtgcacaacgccaagaccaagcctcgcgaggagcagttcaactccacctaccgcgtggtgtccgtgctgaccgtgctgcaccaggactggctgaacggcaaggagtacaagtgcaaggtgtccaacaagggcctgccttcctccatcgagaagaccatctccaaggccaagggccagcctcgcgagcctcaggtgtacaccctgcctccttcccaggaggagatgaccaagaaccaggtgtccctgacctgcctggtgaagggcttctacccttccgacatcgccgtggagtgggagtccaacggccagcctgagaacaactacaagaccacccctcctgtgctggactccgacggctccttcttcctgtactcccgcctgaccgtggacaagtcccgctggcaggagggcaacgtgttctcctgctccgtgatgcacgaggccctgcacaaccactacacccagaagtccctgtccctgtccctgggcaagtaagtcgac

SEQ ID NO: 76 - protein, 460 aa, Artificial sequence
  An amino acid sequence of heavy chain (BY18.1H) of anti-PD1 antibody BY18.1
METDTLLLWVLLLWVPGSTGQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - DNA, 1113 bp, Artificial sequence
  A nucleotide sequence of fusion protein BY31.19 (CD80-Fc, IgG4)
ctcgaggccaccatggagaccgacacactcctcctgtgggtgctgctgctgtgggtgcctggctccactggcgtcatccatgtgaccaaagaggtcaaagaagtcgcaactctgtcttgcggacacaacgtctccgtcgaagaactcgcccagactagaatatattggcagaaggaaaagaagatggtgctcaccatgatgtccggcgatatgaacatttggcctgagtacaagaaccggacaatcttcgatattactaataacctgagtatcgtgattctcgctctgcgccctagcgacgagggcacatacgagtgtgtggtgctgaagtacgagaaggatgcattcaagcgggagcacctcgcagaggtgacactctccgtgaaggccgacttcccaaccccatctgacaagcgcgtggagtccaagtacggccctccttgccctccttgccctgcccctgagttcctgggcggcccttccgtgttcctgttccctcctaagcctaaggacaccctgatgatctcccgcacccctgaggtgacctgcgtggtggtggacgtgtcccaggaggaccctgaggtgcagttcaactggtacgtggacggcgtggaggtgcacaacgccaagaccaagcctcgcgaggagcagttcaactccacctaccgcgtggtgtccgtgctgaccgtgctgcaccaggactggctgaacggcaaggagtacaagtgcaaggtgtccaacaagggcctgccttcctccatcgagaagaccatctccaaggccaagggccagcctcgcgagcctcaggtgtacaccctgcctccttcccaggaggagatgaccaagaaccaggtgtccctgacctgcctggtgaagggcttctacccttccgacatcgccgtggagtgggagtccaacggccagcctgagaacaactacaagaccacccctcctgtgctggactccgacggctccttcttcctgtactcccgcctgaccgtggacaagtcccgctggcaggagggcaacgtgttctcctgctccgtgatgcacgaggccctgcacaaccactacacccagaagtccctgtccctgtccctgggctaagaattc

SEQ ID NO: 78 - protein, 364 aa, Artificial sequence
  An amino acid sequence of the fusion protein BY31.19 (CD80-Fc, IgG4)
METDTLLLWVLLLWVPGSTGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 79 - DNA, 723 bp, Artificial sequence
  A nucleotide sequence of the first subunit (BY24.4L) of the fusion protein BY24.4 (kappa, IgG4)
ctcgaggccaccatggagacagatactctgctgctctgggtcctgctgctgtgggtgccaggcagcacaggcgacatccagatgacccagtccccttcctccctgtctgcctccgtgggcgacagggtgaccatcacctgttccgcatcccaggatatctccaactacctgaactggtatcagcagaagccaggaaaggcccccaaggtgctgatctactttacttcttccctgcattccggcgtgccctccaggttctctggctccgggtctggaaccgactttacactgacaatctcctctctgcagccagaagactttgccacctattactgccagcagtactccactgtcccctggaccttcgggcaaggaaccaaagtggaaatcaagaggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaagaattc

SEQ ID NO: 80 - protein, 234 aa, Artificial sequence
  An amino acid sequence of the first subunit (BY24.4L) of the fusion protein BY24.4 (kappa, IgG4)
METDTLLLWVLLLWVPGSTGDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - DNA, 1806 bp, Artificial sequence
  A nucleotide sequence of the second subunit (BY24.4H) of the fusion protein BY24.4 (kappa, IgG4)
tctagagccaccatggaaaccgacacactgctgctgtgggtcctgctgctgtgggtgccaggctctacaggcgaagtgcagctggtcgaatctggcggcggactggtgcaaccagggggcagcctgaggctgtcctgcgccgcttctggatacacctttaccaattacggcatgaattgggtgaggcaggctcctggcaagggcctggagtgggtgggatggatcaacacctacaccggcgagcccacctacgccgctgactttaagaggaggttcaccttctctctggacacctccaagtccaccgcttacctgcaaatgaactctctgagggctgaagacaccgccgtgtactactgcgccaagtacccccattactacgggtcctcccactggtacttcgacgtgtggggccagggcaccctcgtgacagtgtcttccgcctccaccaagggcccttccgtgttccctctggccccttgctcccgctccacctccgagtccaccgccgccctgggctgcctggtgaaggactacttccctgagcctgtgaccgtgtcctggaactccggcgccctgacctccggcgtgcacaccttccctgccgtgctgcagtcctccggcctgtactccctgtcctccgtggtgaccgtgccttcctcctccctgggcaccaagacctacacctgcaacgtggaccacaagccttccaacaccaaggtggacaagcgcgtggagtccaagtacggccctccttgccctccttgccctgcccctgagttcctgggcggcccttccgtgttcctgttccctcctaagcctaaggacaccctgatgatctcccgcacccctgaggtgacctgcgtggtggtggacgtgtcccaggaggaccctgaggtgcagttcaactggtacgtggacggcgtggaggtgcacaacgccaagaccaagcctcgcgaggagcagttcaactccacctaccgcgtggtgtccgtgctgaccgtgctgcaccaggactggctgaacggcaaggagtacaagtgcaaggtgtccaacaagggcctgccttcctccatcgagaagaccatctccaaggccaagggccagcctcgcgagcctcaggtgtacaccctgcctccttcccaggaggagatgaccaagaaccaggtgtccctgacctgcctggtgaagggcttctacccttccgacatcgccgtggagtgggagtccaacggccagcctgagaacaactacaagaccacccctcctgtgctggactccgacggctccttcttcctgtactcccgcctgaccgtggacaagtcccgctggcaggagggcaacgtgttctcctgctccgtgatgcacgaggccctgcacaaccactacacccagaagtccctgtccctgtccctgggcggcggaggatctggcggcggaggcagtggaggcggcggaagtgtgatccatgtaacaaaagaagtgaaggaagtggctacactctcttgcggccacaacgtgtccgtggaggaactagctcagacccggatctattggcagaaagaaaagaagatggtgctgaccatgatgtccggcgacatgaacatttggccagagtacaagaaccgcacaattttcgacattacaaacaacctctctattgtgattctggctctcaggcctagcgacgagggcacatacgagtgcgtggtgctcaagtacgagaaggacgctttcaagcgggagcacctcgctgaggtgaccctgtccgtgaaggccgacttccctactccatcttaagtcgac

SEQ ID NO: 82 - protein, 595 aa, Artificial sequence
  An amino acid sequence of the second subunit (BY24.4H) of the fusion protein BY24.4 (kappa, IgG4)
METDTLLLWVLLLWVPGSTGEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPS

SEQ ID NO: 83 - DNA, 723 bp, Artificial sequence
  A nucleotide sequence of the first subunit (BY24.5L) of the fusion protein BY24.5 (kappa, IgG4)
ctcgaggccaccatggagacagatactctgctgctctgggtcctgctgctgtgggtgccaggcagcacaggcgacatccagatgacccagtctcctagctccgtgagcgcctctatcggcgacagagtgaccatcacatgcagagcttctcagggcatcgacaactggctcggctggtaccagcagaagcctggcaaggccccaaagctgctgatctacgacgcctctaacctggacaccggcgtgccatcccggttcagcggctctggctccggcacatacttcacactcacaatctcttccctgcaggccgaggacttcgctgtgtacttctgccagcaggctaaggctttcccacctacattcggcggcggaaccaaagtggaaatcaagaggaccgtggccgcccccagcgtgttcatcttccctcccagcgacgagcagctgaagtctggcaccgccagcgtggtgtgcctgctgaacaacttctacccccgcgaggccaaggtgcagtggaaggtggacaacgccctgcagagcggcaacagccaggagagcgtgaccgagcaggactccaaggacagcacctacagcctgagcagcaccctgaccctgagcaaggccgactacgagaagcacaaggtgtacgcctgcgaggtgacccaccagggactgtctagccccgtgaccaagagcttcaaccggggcgagtgctaagaattc

SEQ ID NO: 84 - protein, 234 aa, Artificial sequence
  An amino acid sequence of the first subunit (BY24.5L) of the fusion protein BY24.5 (kappa, IgG4)
METDTLLLWVLLLWVPGSTGDIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - DNA, 1785 bp, Artificial sequence
  A nucleotide sequence of the second subunit (BY24.5H) of the fusion protein BY24.5 (kappa, IgG4)
tctagagccaccatggaaaccgacacactgctgctgtgggtcctgctgctgtgggtgccaggctctacaggcgaggtgcagctcgtgcagtctggcggcggcctcgtgaagccaggcggctccctcagactgtcttgcgctgctagcggcttcacattctcttcctactctatgaactgggtgagacaggctcccggcaagggcctggagtgggtgtccagcatctcttcctctagctcttacatctactacgccgactccgtgaagggcagattcacaatcagccgggacaacgccaagaactccctgtacctgcagatgaacagcctgagagccgaggacaccgctgtgtactactgcgctagagtgaccgacgctttcgacatctggggccagggcactctcgtgacagtgtcttccgcctccaccaagggcccttccgtgttccctctggccccttgctcccgctccacctccgagtccaccgccgccctgggctgcctggtgaaggactacttccctgagcctgtgaccgtgtcctggaactccggcgccctgacctccggcgtgcacaccttccctgccgtgctgcagtcctccggcctgtactccctgtcctccgtggtgaccgtgccttcctcctccctgggcaccaagacctacacctgcaacgtggaccacaagccttccaacaccaaggtggacaagcgcgtggagtccaagtacggccctccttgccctccttgccctgcccctgagttcctgggcggcccttccgtgttcctgttccctcctaagcctaaggacaccctgatgatctcccgcacccctgaggtgacctgcgtggtggtggacgtgtcccaggaggaccctgaggtgcagttcaactggtacgtggacggcgtggaggtgcacaacgccaagaccaagcctcgcgaggagcagttcaactccacctaccgcgtggtgtccgtgctgaccgtgctgcaccaggactggctgaacggcaaggagtacaagtgcaaggtgtccaacaagggcctgccttcctccatcgagaagaccatctccaaggccaagggccagcctcgcgagcctcaggtgtacaccctgcctccttcccaggaggagatgaccaagaaccaggtgtccctgacctgcctggtgaagggcttctacccttccgacatcgccgtggagtgggagtccaacggccagcctgagaacaactacaagaccacccctcctgtgctggactccgacggctccttcttcctgtactcccgcctgaccgtggacaagtcccgctggcaggagggcaacgtgttctcctgctccgtgatgcacgaggccctgcacaaccactacacccagaagtccctgtccctgtccctgggcggcggaggatctggcggcggaggcagtggaggcggcggaagtgtgatccatgtaacaaaagaagtgaaggaagtggctacactctcttgcggccacaacgtgtccgtggaggaactagctcagacccggatctattggcagaaagaaaagaagatggtgctgaccatgatgtccggcgacatgaacatttggccagagtacaagaaccgcacaattttcgacattacaaacaacctctctattgtgattctggctctcaggcctagcgacgagggcacatacgagtgcgtggtgctcaagtacgagaaggacgctttcaagcgggagcacctcgctgaggtgaccctgtccgtgaaggccgacttccctactccatcttaagtcgac

SEQ ID NO: 86 - protein, 588 aa, Artificial sequence
  An amino acid sequence of the second subunit (BY24.5H) of the fusion protein BY24.5 (kappa, IgG4)
METDTLLLWVLLLWVPGSTGEVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPS

SEQ ID NO: 87 - DNA, 1776 bp, Artificial sequence
  A nucleotide sequence of the fusion protein BY24.12 (CD80-Fc-VEGFR)
ctcgaggccaccgccaccatggagaccgacacactcctcctgtgggtgctgctgctgtgggtgcctggctccactggcgtcatccatgtgaccaaagaggtcaaagaagtcgcaactctgtcttgcggacacaacgtctccgtcgaagaactcgcccagactagaatatattggcagaaggaaaagaagatggtgctcaccatgatgtccggcgatatgaacatttggcctgagtacaagaaccggacaatcttcgatattactaataacctgagtatcgtgattctcgctctgcgccctagcgacgagggcacatacgagtgtgtggtgctgaagtacgagaaggatgcattcaagcgggagcacctcgcagaggtgacactctccgtgaaggccgacttcccaaccccatctgacaagcgcgtggagtccaagtacggccctccttgccctccttgccctgcccctgagttcctgggcggcccttccgtgttcctgttccctcctaagcctaaggacaccctgatgatctcccgcacccctgaggtgacctgcgtggtggtggacgtgtcccaggaggaccctgaggtgcagttcaactggtacgtggacggcgtggaggtgcacaacgccaagaccaagcctcgcgaggagcagttcaactccacctaccgcgtggtgtccgtgctgaccgtgctgcaccaggactggctgaacggcaaggagtacaagtgcaaggtgtccaacaagggcctgccttcctccatcgagaagaccatctccaaggccaagggccagcctcgcgagcctcaggtgtacaccctgcctccttcccaggaggagatgaccaagaaccaggtgtccctgacctgcctggtgaagggcttctacccttccgacatcgccgtggagtgggagtccaacggccagcctgagaacaactacaagaccacccctcctgtgctggactccgacggctccttcttcctgtactcccgcctgaccgtggacaagtcccgctggcaggagggcaacgtgttctcctgctccgtgatgcacgaggccctgcacaaccactacacccagaagtccctgtccctgtccctgggcggcggaggatctggcggcggaggcagtggaggcggcggaagtgcttccgacaccggccgccctttcgtggagatgtactccgagatccctgagatcatccacatgaccgagggccgcgagctggtgatcccttgccgcgtgacctcccctaacatcaccgtgaccctgaagaagttccctctggacaccctgatccctgacggcaagcgcatcatctgggactcccgcaagggcttcatcatctccaacgccacctacaaggagatcggcctgctgacctgcgaggccaccgtgaacggccacctgtacaagaccaactacctgacccaccgccagaccaacaccatcatcgacgtggtgctgtccccttcccacggcatcgagctgtccgtgggcgagaagctggtgctgaactgcaccgcccgcaccgagctgaacgtgggcatcgacttcaactgggagtacccttcctccaagcaccagcacaagaagctggtgaaccgcgacctgaagacccagtccggctccgagatgaagaagttcctgtccaccctgaccatcgacggcgtgacccgctccgaccagggcctgtacacctgcgccgcctcctccggcctgatgaccaagaagaactccaccttcgtgcgcgtgcacgagtaagaattc

SEQ ID NO: 88 - protein, 583 aa, Artificial sequence
  An amino acid sequence of the fusion protein BY24.12 (CD80-Fc-VEGFR)
METDTLLLWVLLLWVPGSTGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGSASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHE

(19) United States
(12) Patent Application Publication
(10) Pub. No.: US 2022/0106389 A1
(43) Pub. Date: Apr. 7, 2022

(54) MULTI-TARGETING FUSION PROTEIN FOR BLOCKING THE GROWTH OF VASCULAR ENDOTHELIAL CELLS AND ACTIVATING T CELLS AND PHARMACEUTICAL COMPOSITION COMPRISING THE SAME

(71) Applicants: Beijing Beyond Biotechnology Co., Ltd, Beijing (CN); Lunan Pharmaceutical Group Corporation, Linyi, Shandong (CN)

(72) Inventors: Pinliang Hu, Beijing (CN); Jing Zou, Beijing (CN); Weidong Hong, Beijing (CN); Yun He, Beijing (CN); Jie Bai, Beijing (CN); Lingyun Song, Beijing (CN); Wendi Yang, Beijing (CN); Guimin Zhang, Linyi (CN); Lili Zhao, Linyi (CN); Zhong Liu, Linyi (CN); Zhenyu Li, Linyi (CN); Zhongsong Zhu, Linyi (CN)

(21) Appl. No.: 17/421,874

(22) PCT Filed: Jan. 9, 2020
(86) PCT No.: PCT/CN2020/071213

§ 371 Date: Jul. 9, 2021

(30) Foreign Application Priority Data
Jan. 10, 2019 (CN) 201910023522.6

(51) Int. Cl. C07K 16/22; C07K 14/705; C07K 16/28; C07K 14/71; A61P 35/00

(52) U.S. Cl. CPC C07K 16/22; C07K 14/70532; C07K 16/2863; C07K 14/71; A61K 2039/505; A61P 35/00; C07K 2319/30; C07K 2317/55; C07K 2317/92; C07K 16/2818

(57) ABSTRACT

The present invention provides a multi-targeting fusion protein that blocks the growth of vascular endothelial cells and activates T cells, which comprises (i) a vascular endothelial cell growth inhibitory domain; (ii) an immunoglobulin Fc domain; and (iii) CD80 extracellular domain (ECD). The present invention also provides a polynucleotide encoding the multi-targeting fusion protein, a vector containing the polynucleotide, a host cell comprising the polynucleotide or the vector, and pharmaceutical composition comprising the multi-targeting fusion protein and an anti-PD-1 antibody. The multi-targeting fusion protein and pharmaceutical composition of the present invention can treat or prevent cancerous diseases in individuals.

### FIELD OF TECHNOLOGY

The present invention generally relates to the field of medical biotechnology. Specifically, the present invention relates to a multi-targeting fusion protein comprising (i) a vascular endothelial cell growth inhibitor domain; (ii) an immunoglobulin Fc domain; and (iii) a multi-targeting fusion protein of CD80 extracellular domain (ECD), a polynucleotide encoding the multi-targeting fusion protein, a vector containing the polynucleotide, a host cell comprising the polynucleotide or the vector, and pharmaceutical composition comprising the multi-targeting fusion protein and an anti-PD-1 antibody. The multi-targeting fusion protein and pharmaceutical composition of the present invention can treat or prevent cancerous diseases in individuals.

### BACKGROUND

Tumor microenvironment is a complex environment for tumor cells to survive and develop and it is composed of cellular components and non-cellular components, wherein cellular components include tumor cells, immune cells, endothelial cells, etc.; non-cellular components include cytokines, chemokines, and the like. With the research on tumors, people have realized that the generation, growth and metastasis of tumors are regulated by the tumor microenvironment. The tumor microenvironment determines whether tumor cells can grow dominantly

Inhibition of T lymphocyte activity usually exists in the tumor microenvironment, resulting in the T lymphocytes unable to effectively exert a killing effect on tumors (Yao S, Zhu Y and Chen L., Advances in targeting cell surface signaling molecules for immune modulation., Nat Rev Drug Discov, 2013, 12(2):130-146), for example, tumor cells inhibit the activity of T lymphocytes by expressing PD-L1, thereby using the inhibitory signaling pathway of immune checkpoints (i.e., PD-1/PD-L1 inhibitory signal pathway), wherein the PD-L1 is not expressed in normal human tissues.

It was previously thought that inhibition of T lymphocyte activation is achieved by inhibition of the T cell receptor (TCR)/major histocompatibility complex (MHC) after binding of PD-1 to its ligand PD-L1, but recent studies by Hui et al. have shown that PD-1 and CD 28 co-localize to the T lymphocyte membrane, targets of PD-1 mediated immunosuppression are mainly CD 28 rather than TCR. Specifically, Shp2 phospholipase is rapidly recruited after PD-1 binds to PD-L1 and Shp2 phospholipase preferentially dephosphorylates CD28, which is stronger than dephosphorylation of TCR, thereby inhibiting T cell function by inactivating the signal transduction of CD28. (Hui E. https://www.ncbi.nlm.nih.gov/pubmed/?term=Hui %20E %5bAuthor %5d&cauthor=true&cauthor uid=2828024 7 et al., T cell costimulatory receptor CD28 is a primary target for PD-1-mediated inhibition, Science, https://www.ncbi.nlm.nih.gov/pubmed/?term=Enfu+Hui+Jeanne+Cheung 2017, 355 (6332): 1428-1433).

Kamphorst A O et al. confirmed that the activation of CD28 costimulatory signals is one of the important conditions for T cell “reactivation” (Kamphorst A O et al., Rescue of exhausted CD8 T cells by PD-1-targeted therapies is CD28-dependent, Science, 2017, 355(6332): 1423-1427). The activation of the costimulatory pathway of CD28/B7 (that is, the costimulatory molecule CD28 on the surface of T lymphocytes combined with CD86 (also known as B7-2) or CD80 (also known as B7-1)) is critical to tumor-bearing mice and the therapeutic effect of anti-PD-1 antibody during chronic viral infection. If the binding of B7 molecules to CD28 is blocked with anti-B7 antibodies, the inhibition of the anti-PD-1 antibody against the tumor is significantly reduced. Other studies have shown that soluble CD80 has therapeutic effects in vivo in mouse tumor systems (Horn L A et al., Soluble CD80 Protein Delays Tumor Growth and Promotes Tumor-Infiltrating Lymphocytes, Cancer Immunol Res. 2018; 6(1): 59-68). The low or no expression of CD80 and CD86 in the tumor microenvironment is also one of the important mechanisms that cause tumor immune evasion.

In addition, CTLA-4 expressed on the surface of T cells and the costimulatory molecule CD28 on the surface of T cells have a high degree of homology, and they have the same ligand CD86 (B7-2) or CD80 (B7-1). The combination of CTLA-4 and B7 molecules usually inhibits the activation of T cells, thus blocking the immune checkpoint B7/CTLA-4 pathway may enhance tumor-specific T cell activation.

On the other hand, tumor cells also release pro-angiogenic factors in the tumor microenvironment, such as Vascular Endothelial Cell Growth Factor (VEGF), which leads to a surge in the number of VEGF, VEGF mediates the proliferation and migration of vascular endothelial cells, improves vascular permeability, inhibits tumor cell apoptosis and provides a good microenvironment for tumor growth and metastasis by binding to its cell surface receptor VEGFR.

Currently, single-target tumor therapeutic drugs for blocking the PD-1/PD-L1 inhibitory signaling pathway, blocking the B7/CTLA-4 pathway, or blocking the VEGF/VEGFR pathway have been approved for marketing. They are, for example, anti-PD-1 antibody drugs, such as Nivolumab from Bristol-Myers Squibb Company (BMS) and Pembrolizumab from Merck Company (Merck); CTLA-4 antibody Ipilimumab (Ipilimumab, trade name Yervoy) from Bristol-Myers Squibb Company (BMS); Human-mouse chimeric anti-VEGF antibody Bevacizumab (trade name Avastin) from Genentech Inc.; Aflibercept Developed as a VEGF-Trap by Sanofi-aventis Company and Regeneron Company and so on. However, a considerable number of cancer patients do not respond to single-targeting tumor therapy or develop drug resistance. For example, the current average treatment efficiency of anti-PD-1 and PD-L1 antibody drugs is only about 20%, and the five-year survival rate of lung cancer is only 16%. Therefore, in many cases, multi-targeting therapy needs to be employed to avoid the problem that patients have no response to a single targeted tumor or generate drug resistance.

Since multi-targeting fusion proteins are capable of simultaneously specifically targeting multiple Signal transduction pathways involved in tumor generation and development, there is a need to develop multi-targeting fusion proteins capable of improving tumor microenvironment by blocking vascular endothelial cell growth and activating T cells, and the need to combine the multi-targeting fusion protein with other anti-cancer drugs.

### SUMMARY OF THE INVENTION

The present inventors have developed a set of multi-targeting fusion proteins that block growth of vascular endothelial cells and activate T cells by the inventor (s), which comprises (i) a vascular endothelial cell growth inhibitor domain; (ii) an immunoglobulin FC domain; and (iii) a CD80 extracellular domain (ECD). The multi-targeting fusion protein can improve the tumor microenvironment and the effect of tumor immunotherapy from two aspects, wherein the improvement on the tumor microenvironment on one hand is achieved by the specific binding of the CD80 extracellular domain (ECD) of the multi-targeting fusion protein to CD28, PD-L1 and CTLA-4, specifically PD-1/PD-L1 inhibitory signal pathway is relieved to “Brake release” the immune system by binding PD-L1 this immune checkpoint to the CD80 extracellular domain; the same function of inhibiting regulatory T cells (Treg) as the anti-CTLA-4 antibody ipilimumab is exerted by binding the CD80 extracellular domain to CTLA-4; the CD28/B7 costimulatory pathway is activated through binding the CD80 extracellular domain to CD28 and T lymphocytes are activated to “throttle up the engine” for the activation of lymphocytes; on the other hand, the improvement of the tumor microenvironment is achieved by blocking the VEGF/VEGFR pathway through the vascular endothelial cell growth inhibitor domain of the multi-targeting fusion protein to inhibit the formation of tumor angiogenesis and to normalize tumor tissue blood vessels, so that more lymphocytes invade the tumor tissue, and relieve the inhibitory effect of VEGFs on immune cells.

In one embodiment, the multi-targeting fusion protein of the present invention comprises (i) derived from an antigen-binding fragment of an anti-VEGF antibody and/or an anti-VEGFR antibody and/or a VEGFR extracellular receptor functional domain; (ii) an immunoglobulin Fc domain; and (iii) a CD80 extracellular domain (ECD).

The antigen-binding fragment derived from the anti-VEGF antibody contained in the multi-targeting fusion protein can be derived from an antigen-binding fragment of any anti-VEGF antibody, as long as it is an antibody that can bind to VEGF and thereby block or inhibit the binding of VEGF to its receptor VEGFR. The anti-VEGF antibodies include anti-VEGF antibodies known in the prior art and anti-VEGF antibodies developed in the future. In one embodiment, preferably, the antigen-binding fragment of the anti-VEGF antibody is the Fab, Fab′, F(ab′)2, Fv, single-chain Fv of the anti-VEGF antibody; preferably, the antigen-binding fragment of the anti-VEGF antibody comprises all six heavy chain CDRs and light chain CDRs that are contained in paired heavy chain variable region sequence/light chain variable region sequence selected from the group consisting of SEQ ID NO: 1/2, 3/4, and 5/6, or a sequence having one, two, three, four, or five amino acid changes (e.g., amino acid replacement or deletion) from one or more CDR(s) in all the six heavy chain CDRs and light chain CDRs; more preferably, the antigen-binding fragment of the anti-VEGF antibody comprises the paired heavy chain variable region sequence/light chain variable region sequence selected from the group consisting of SEQ ID NO: 1/2, 3/4 and 5/6, or a sequence having at least 90%, 91%, 92%, 93%, 94%, 95%, 96%, 97%, 98%, 99% or more sequence identity to the paired heavy chain variable region sequence/light chain variable region sequence; most preferably, the antigen-binding fragment of the anti-VEGF antibody is the Fab of Bevacizumab, Ranibizumab or Ramucirumab.

The antigen-binding fragment derived from the anti-VEGFR antibody contained in the multi-targeting fusion protein can be derived from an antigen-binding fragment of any anti-VEGFR antibody, as long as it is an antibody that can bind to VEGFR and thereby block or inhibit VEGF binding to its receptor VEGFR. The anti-VEGFR antibodies include anti-VEGFR antibodies known in the art and anti-VEGFR antibodies developed in the future. In one embodiment, preferably, the antigen-binding fragment of the anti-VEGFR antibody is the Fab, Fab′, F(ab′)2, Fv, single-chain Fv of the anti-VEGFR antibody and/or the anti-VEGFR antibody; preferably, the antigen-binding fragment of the anti-VEGFR antibody comprises all six heavy chain CDRs and light chain CDRs that are contained in paired heavy chain variable region sequence/light chain variable region sequence selected from the group consisting of SEQ ID NO: 1/2, 3/4, and 5/6, or a sequence having one, two, three, four, or five amino acid changes (e.g., amino acid replacement or deletion) from one or more CDR(s) in all the six heavy chain CDRs and light chain CDRs; more preferably, the antigen-binding fragment of the anti-VEGFR antibody comprises the paired heavy chain variable region sequence/light chain variable region sequence selected from the group consisting of SEQ ID NO: 1/2, 3/4 and 5/6, or a sequence having at least 90%, 91%, 92%, 93%, 94%, 95%, 96%, 97%, 98%, 99% or more sequence identity to the paired heavy chain variable region sequence/light chain variable region sequence; most preferably, the antigen-binding fragment of the anti-VEGFR antibody is the Fab of Bevacizumab, Ranibizumab or Ramucirumab;

The VEGFR extracellular receptor functional domain contained in the multi-targeting fusion protein can be any VEGFR extracellular receptor functional domain, provided that it is an VEGFR extracellular receptor functional domain that can bind to VEGF and thereby block or inhibit the binding of VEGF to its receptor VEGFR. Preferably, the VEGFR extracellular receptor functional domain comprises the immunoglobulin-like domain 2 of VEGFR1 and the immunoglobulin-like domain 3 of VEGFR2; or the VEGFR extracellular receptor functional domain comprises the immunoglobulin-like domain 2 of VEGFR1 and the immunoglobulin-like domain 3 of VEGFR2 and the immunoglobulin-like domain 4 of VEGFR2; or the VEGFR extracellular receptor functional domain comprises the immunoglobulin-like domain 2 of VEGFR1; more preferably, the VEGFR extracellular receptor functional domain has any amino acid sequence selected from SEQ ID NO: 7-9 or an amino acid sequence having at least 90%, 91%, 92%, 93%, 94%, 95%, 96%, 97%, 98%, 99% or more sequence identity to the amino acid sequence shown in SEQ ID NO: 7-9.

(ii) The immunoglobulin Fc domain contained in the multi-targeting fusion protein can be any immunoglobulin Fc domain, in particular, the (ii) is a human immunoglobulin Fc domain. In one embodiment, the immunoglobulin Fc domain is an Fc domain of an IgG class antibody, in particular an Fc domain of IgG₁ subclass, IgG₂ subclass, IgG₄ subclass antibody. In a preferred embodiment, the immunoglobulin Fc domain contained in the multi-targeting fusion protein of the present invention is an Fc domain of an IgG1 subclass antibody, in particular an Fc domain of a human IgG1 subclass antibody. In a preferred embodiment, the immunoglobulin Fc domain contained in the multi-targeting fusion protein of the present invention is an Fc domain of an IgG4 subclass antibody, in particular an Fc domain of a human IgG4 subclass antibody. In some embodiments, (ii) the immunoglobulin Fc domain of the multi-targeting fusion protein of the present invention comprises an Fc domain of the amino acid sequence shown in SEQ ID NO: 10, 11, or 12, or an Fc domain comprising at least 90%, 91%, 92%, 93%, 94%, 95%, 96%, 97%, 98%, 99%, or more sequence identity to the amino acid sequence shown in SEQ ID NO: 10, 11, or 12.

(III) The CD80 ECD included in the multi-targeting fusion protein is part of the extracellular domain of CD80. In one embodiment, the CD80 ECD comprises a CD80 immunoglobulin V (IgV) domain (CD80-IgV). In one embodiment, the CD80 ECD comprises a CD80 immunoglobulin V domain and a C domain (CD80-IgVIgC). In one embodiment, the CD80 ECD is a human CD80 ECD, preferably the CD80 ECD comprises a human CD80 IgV. In one embodiment, the CD80-IgV has an amino acid sequence as shown in SEQ ID NO: 13, or an amino acid sequence having at least 90%, 91%, 92%, 93%, 94%, 95%, 96%, 97%, 98%, 99% or more identity to the amino acid sequence of SEQ ID NO: 13. In one embodiment, the CD80-IgVIgC has an amino acid sequence as shown in SEQ ID NO: 14, or an amino acid sequence having at least 90%, 91%, 92%, 93%, 94%, 95%, 96%, 97%, 98%, 99% or more identity to the amino acid sequence of SEQ ID NO: 14.

In one embodiment, the multi-targeting fusion protein further comprises a peptide linker between the (i), (ii), and/or (iii); preferably, the peptide linker comprises one or more amino acids, more preferably at least 5 amino acids, and most preferably comprises a peptide linker selected from the group consisting of SEQ ID NOs: 20-46.

In one embodiment, the multi-targeting fusion protein is effectively linked from the n-terminal to the c-terminal in the order of (i), (ii), and (iii); in the order of (iii), (i) and (ii); or in the order of (iii), (ii), (i).

In some embodiments, the multi-targeting fusion protein of the present invention comprises

(a) A full-length anti-VEGF antibody, a full-length anti-VEGFR antibody, or a full-length anti-VEGF and VEGFR bispecific antibody; and one CD80 ECD effectively linked to the C-terminal of each of the two heavy chains of the antibody;

(b) A full-length anti-VEGF antibody, a full-length anti-VEGFR antibody, or a full-length anti-VEGF and VEGFR bispecific antibody; and one CD80 ECD effectively linked to the N-terminal of each of the two heavy chains of the antibody; and one CD80 ECD effectively connected to the N-terminal of each of the two light chains of the antibody;

(c) A CD80 ECD; an immunoglobulin Fc domain in dimer form effectively linked at the C-terminal of a CD80 ECD; and an antigen-binding fragment derived from an anti-VEGF antibody and/or an anti-VEGFR antibody effectively connected at the C-terminal of an immunoglobulin Fc domain in dimer form;

or

(d) A CD80 ECD; an immunoglobulin Fc domain in dimer form effectively linked at the C-terminal of a CD80 ECD; and a VEGFR extracellular receptor functional domain effectively connected to the C-terminal of the immunoglobulin Fc domain in dimer form;

Preferably, the antibody is an IgG class antibody, particularly an IgG₁ subclass, an IgG₂ subclass, an IgG₄ subclass antibody; and more particularly an IgG4 subclass antibody; also preferably, the IgG₄ subclass antibody contains amino acid replacement at the S228 position of the Fc domain, more preferably amino acid replacement S228P; further preferably, the light chain type of the antibody is κ type or λ type, preferably κ type;

Preferably, the full-length anti-VEGF antibody is Bevacizumab, and the full-length anti-VEGFR antibody is Ramucirumab.

In some embodiments, the multi-targeting fusion protein of the present invention is selected from

(1) A fusion protein comprising the first subunit of the fusion protein of SEQ ID NO: 80 and the second subunit of the fusion protein of SEQ ID NO: 82;

(2) A fusion protein comprising the first subunit of the fusion protein of SEQ ID NO: 84 and the second subunit of the fusion protein of SEQ ID NO: 86;

(3) A fusion protein comprising the fusion protein subunit of SEQ ID NO:88.

The present invention also provides polynucleotides encoding multi-targeting fusion proteins of the present invention, vectors comprising polynucleotides encoding multi-targeting fusion proteins of the present invention, preferably expression vectors, and most preferably glutamine synthetase expression vectors with double expression cassettes. In another aspect, the present invention provides a host cell comprising a polynucleotide or vector of the present invention. In one embodiment, the host cell is CHO, HEK293, or NSO cell. The present invention also provides a method for producing a multi-targeting fusion protein of the present invention, which comprises step (i) culturing host cell of the present invention under conditions suitable for expressing the multi-targeting fusion protein of the present invention, and step (ii) recovering the multi-targeting fusion protein of the present invention.

In another aspect, the invention also provides a pharmaceutical composition of the multi-targeting fusion protein of the present invention in combination with an anti-PD-1 antibody. The multi-targeting fusion protein of the present invention exhibits better synergy when used in combination with an anti-PD-1 antibody, thereby it is possible to achieve the purpose of better inhibition of tumor growth.

When used in combination with the multi-targeting fusion protein of the present invention, the anti-PD-1 antibody can be any anti-PD-1 antibody, as long as it is an antibody capable of inhibiting or reducing the binding of PD-1 to its ligand, including anti-PD-1 antibodies known in the art and anti-PD-1 antibodies developed in the future. In some embodiments, the anti-PD-1 antibody comprises all six heavy chain CDRs and light chain CDRs contained in paired heavy chain variable region sequences/light chain variable region sequence selected from the group consisting of SEQ ID NOS: 47/48, 49/50, 51/52, 53/54, 55/56, 57/58, 59/60, 61/62, 63/64, 65/66, 67/68, 69/70, and 71/72, or a sequence having one, two, three, four, or five amino acid changes (e.g., amino acid replacement or deletion) from one or more CDR(s) of the six heavy chain CDRs and light chain CDRs) sequence; further preferably, the anti-PD-1 antibody comprises paired heavy chain variable region sequence/light chain variable region sequence selected from SEQ ID NO: 47/48, 49/50, 51/52, 53/54, 55/56, 57/58, 59/60, 61/62, 63/64, 65/66, 67/68, 69/70 and 71/72, or a sequence having at least 90%, 91%, 92%, 93%, 94%, 95%, 96%, 97%, 98%, 99% or more sequence identity to the paired heavy chain variable region sequence/light chain variable region sequence; most preferably, the anti-PD-1 antibody is selected from Nivolumab, Pidilizumab and Pembrolizumab.

In yet another aspect, the present invention provides a use of multi-targeting fusion proteins or a use of pharmaceutical compositions of the present invention for manufacturing a medicament for the treatment or prevention of a cancerous disease (e.g., a solid tumor or a soft tissue tumor) in an individual, preferably the cancerous disease is melanoma, breast cancer, colon cancer, esophageal cancer, gastrointestinal stromal tumor (GIST), kidney cancer (e.g., renal cell carcinoma), liver cancer, non-small cell lung cancer (NSCLC), ovarian cancer, pancreatic cancer, prostate cancer, head and neck tumors, gastric cancer, or a hematological malignancy (e.g., lymphoma); in particular, the disease is colon cancer or triple negative breast cancer; preferably, wherein the individual is a mammal, more preferably a human.

Unless otherwise defined, all technical and scientific terms used herein have the same meaning as commonly understood by those ordinary skilled in the art to which the present invention belongs. All publications, patent applications, patents, and other references mentioned herein are hereby incorporated by reference in their entirety. Moreover, the materials, methods, and examples described herein are illustrative only and are not intended to be limiting. Other features, objects, and advantages of the invention will be apparent from the description and drawings, and from the appended claims.

### BRIEF DESCRIPTION OF THE DRAWINGS

The following detailed description of the preferred embodiments of the invention will be better understood when read in conjunction with the following drawings. For the purpose of illustrating the invention, embodiments are shown in the drawings which are presently preferred. However, it is to be understood that the invention is not limited to the precise arrangements and instrumentalities of the embodiments shown in the figures.

FIGS. 1A and B provide a schematic diagram of a multi-targeting fusion protein of the present invention, wherein FIG. 1A illustrates a structural schematic of a multi-targeting fusion protein comprising an antigen-binding fragment of an antibody, an immunoglobulin Fc domain, and a CD80 ECD from an N-terminal to a C-terminus; FIG. 1B illustrates a structural schematic of a fusion protein comprising CD80 ECD, immunoglobulin Fc domain, and VEGFR extracellular receptor functional region from N-terminus to C-terminus.

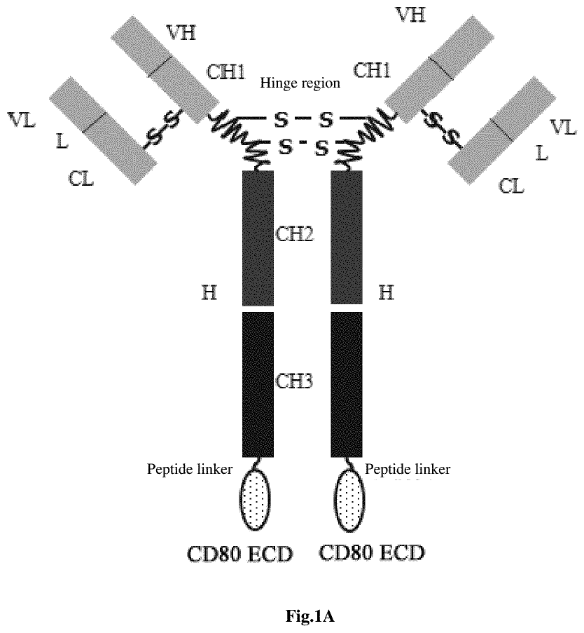

FIG. 2 shows the result that each prepared and purified protein of interest in the present invention is electrophoresed by the SDS-PAGE and then is stained with Coomassie Blue in the presence of a reducing agent (5 mM 1,4-dithiothreitol) in example 2. Lane 1: Protein Molecular Weight Standard Marker; Lane 2: Fusion Protein BY 24.4; Lane 3: Fusion Protein BY 24.5; Lane 4: Fusion Protein BY 24.12; Lane 5: Fusion Protein BY 31.19; Lane 6: Antibody BY 18.1

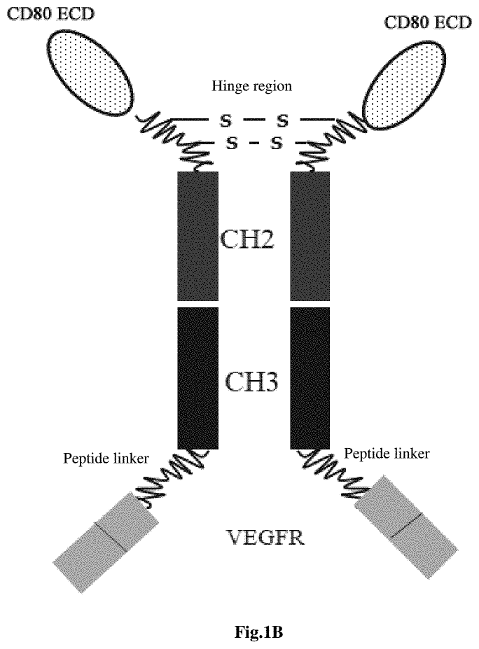

FIG. 3 shows that the multi-targeting fusion protein of the present invention and its combination with anti-PD-1 antibody have effect on the secretion of IFN-γ in the mixed lymphocyte reaction (MLR) in example 5. Column 1: BY18.1 group; column 2: BY24.4 group; column 3: BY18.1+BY24.4 group; column 4: BY24.5 group; column 5: BY18.1+BY24.5 group; column 6: BY24.12 group; column 7: BY18.1+BY24.12 group.

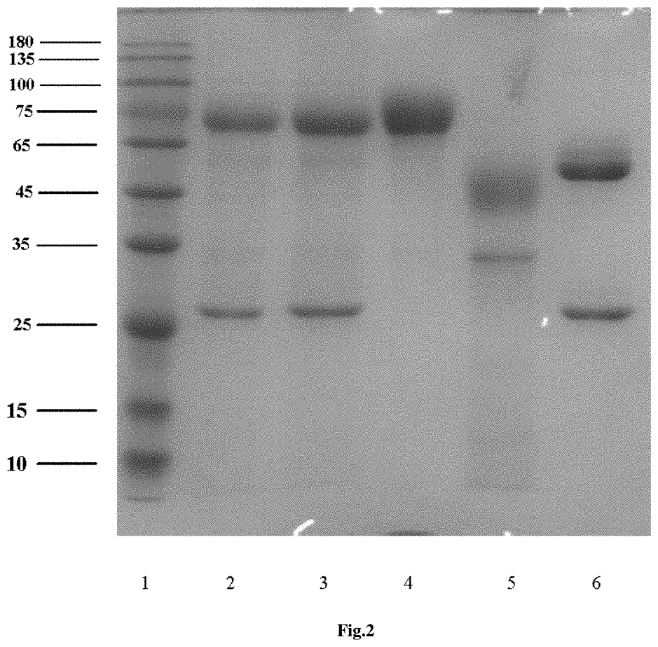

FIG. 4 shows that the multi-targeting fusion protein of the present invention and its combination with anti-PD-1 antibody have anti-tumor growth inhibitory effect on the animal model in Example 6.

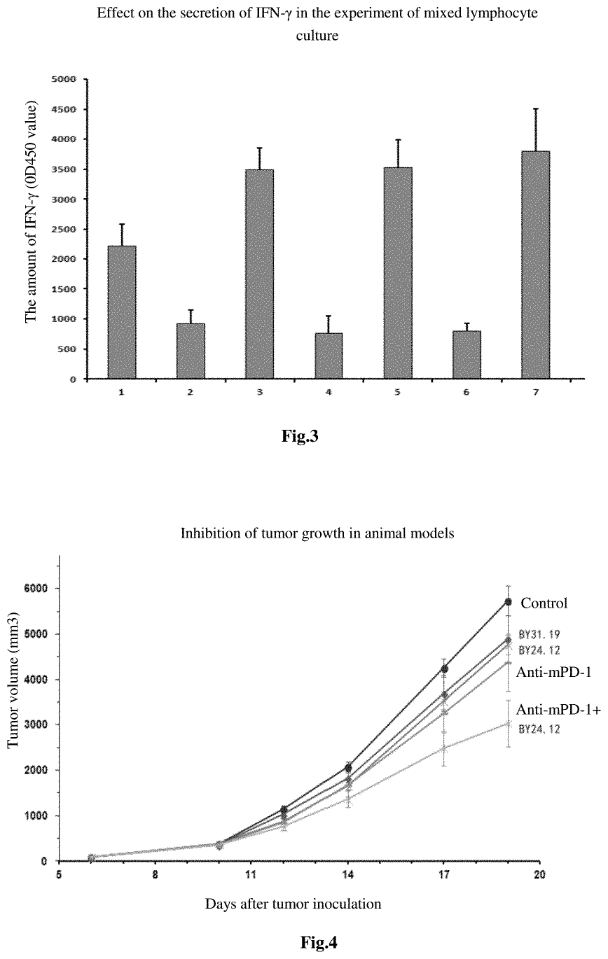

### DETAILED DESCRIPTION OF THE INVENTION

The present invention provides a multi-targeting fusion protein that blocks vascular endothelial cell growth and activates T cells and a pharmaceutical composition comprising the multi-targeting fusion protein. The present invention also provides methods for producing the multi-targeted fusion protein, and the use of the multi-targeting fusion protein or pharmaceutical composition in the treatment or prevention of cancerous disease in individuals.

Unless otherwise defined herein, the terms in this specification are used as commonly used in the art.

### I. Definition

The term “about”, when used in conjunction with a numerical value, is intended to encompass numerical values having a lower limit of 5% less than the specified numerical value and an upper limit of 5% greater than the specified numerical value.

As used herein, the terms “comprises” or “comprising” is intended to include the stated elements, integers, or steps, but not to exclude any other elements, integers, or steps.

The “PD-1/PD-L1 inhibitory signaling pathway”, “PD-1/PD-L1 signal pathway”, “PD-1/PD-L1 signal transduction pathway”, “PD-1/PD-L1 pathway” may be used interchangeably herein to refer to any intracellular signaling pathway initiated by PD-1 binding to PD-L1.

As used herein, “mitigating”, “interfering”, “inhibiting” or “blocking” PD-1/PD-L1 inhibitory signaling pathway may be used interchangeably, meaning (i) interfering with the interaction between PD-1 and PD-L1; and/or (ii) inhibition of at least one biological function leading to a PD-1/PD-L1 signaling pathway. The “mitigating”, “interfering”, “inhibiting” or “blocking” PD-1/PD-L1 signaling pathways resulting from the specific binding of the multi-targeting fusion protein and PD-L1 of the present invention need not be complete mitigating, interfering, inhibiting, or blocking.

As used herein, “CD28/B7 signaling pathway”, “CD28/B7 costimulatory pathway”, “CD28/B7 pathway” may be used interchangeably to refer to (i) a signaling pathway that stimulates cell activation by binding CD28 to CD80; and/or (ii) a signaling pathway that stimulates cell activation by binding CD28 to CD86.

Both “CD80” and “CD86” are transmembrane glycoprotein, which belong to an immunoglobulin superfamily member of a highly similar structure, also referred to as B7 molecules. The CD80 and CD86 extracellular regions are comprised of an immunoglobulin V (IgV) domain and an immunoglobulin C (IgC) domain. The mature CD80 molecule consists of 254 amino acids, wherein the extracellular domain consists of 208 amino acids, 25 amino acids across the membrane domain, and 21 amino acids in the intracellular domain. Similarly, the mature CD86 molecule is composed of 303 amino acids, wherein the extracellular domain is composed of 222 amino acids, 20 amino acids across the transmembrane domain, and 61 amino acids in the intracellular domain. CD80, also known as B7.1, expressed in the surface of T cells, B cells, dendritic cells, and monocytes, binding to CD28, PD-L1 and CTLA-4 at a lower affinity by its immunoglobulin V (IgV) domain, wherein the binding affinity of CD80 to CD28 is 4 μm; binding affinity of CD80 to PD-L1 is-1.7 μm; binding affinity of CD80 to CTLA-4 is 0.2 μm (Butte, J et al. Programmed Death-1 Ligand1 Interaction Specifications with the B7-1 Costimulatory Molecule to Gbit T Cell Responses, Immunity, July 2007; 27 (1): 111-122). CD 86 binds to CD28 and CTLA-4, but does not bind to PD-L1.

Soluble CD80 (eg, CD80-Fc) can produce continuously activate T lymphocytes by a CD28/B7 costimulatory pathway and stimulate production of interferon. Experiments show that CD80-Fc maintains T lymphocytes in vitro to produce interferon, even more efficient than anti-PD-1 antibodies or anti-PD-L1 antibodies. In terms of inhibiting tumor growth in vivo, soluble CD80 (for example, CD80-Fc) is more effective than anti-PD-L1 antibody (Ostrnd-Rosenberg S et al., Novel Strategies for Inhibiting PD-1 Pathway-Mediated Immune Suppression while Simultaneous Delivering Activity Signals to Tumor-Reactive T Cells, Cancer Immunol Immunother. October 2015; 64 (10): 1287-93). The CD80-Fc can inhibit the immunosuppression mediated by PD-1/PD-L1 pathway by binding PD-L1, and deliver a costimulatory signal to T cells activated by the CD28/B7 costimulatory pathway, thereby enhancing the activation of T lymphocyte. In summary, CD80-Fc can mitigate immunosuppression of PD-1/PD-L1 pathways while activating tumor immunoreactive T cells. Although soluble CD86 (eg, CD86-Fc) can also activate CD28, even a 3-5 times activation of CD80-Fc can be produced, but since CD86 does not bind to PD-L1, finally, CD80-Fc has stronger activation effect on T lymphocytes than CD86-Fc (Haile St et al. Soluable CD80 Restorres T Cell Activation and Overcomes Tumor Cell Programmed Death Ligand 1-Mediated Immune Suppression). J Immunol. Sep. 1, 2013; 191 (5): 2829-36). In accordance with existing studies, CD80-Fc has the following effects: (i) when CD80-Fc is used alone, the effect of inhibiting the tumor is better than that of PD-L1 antibody (AACR ANNUAL MEETING, Apr. 14-18, 2018, USA, Ill. Chicago); (ii) CD80-Fc promotes lymphocyte invasion of tumor tissue, and is better than PD-L1 antibody (Horn Lala et al.), Soluble CD80 Protein Delay S Tumor Growth and Promoter Tumor-Infiltrazol, Cancer Immunol Res., January 2018; 6 (1): 59-68); (iii) when CD80-Fc is used alone, the effect of inhibiting the tumor is better than that of the PD-1/PD-L1 pathway and has a synergistic effect when combined with PD-1 antibody. Five-Prime Corporation even considers CD80-Fc superior to T cell agonists such as GITRL, OX40L, and 4-1 BBL. Since the CD80-Fc good immunotherapy effect was seen, the CD80-Fc Project FPT 155 Plan of Five-Prime Therapeutics, Inc. is about to start clinical trials recently.

As used herein, “B7/CTLA-4 pathway”, “B7/CTLA-4 signaling pathway” may be used interchangeably to refer to (i) signaling pathways resulting from binding CD80 to CTLA-4; and/or (ii) signaling pathways resulting from binding CD86 to CTLA-4.

As used herein, “VEGF/VEGFR pathway”, “VEGF/VEGFR signaling pathway” may be used interchangeably to refer to signaling pathways through binding one or more of the VEGF families to one or more of the cell surface receptor VEGFR families. The VEGF family contains six closely related polypeptides, which are highly conserved homodimer glycoprotein, with six subtypes: VEGF-A, -B, -C, -D, -E, and placental growth factor (PLGF), with molecular weights ranging from 35 to 44 kDa. The expression of VEGF-A (including splices thereof such as VEGF 165) is related to the microvascular density of some solid tumors, and the concentration of VEGF-A in the tissue is related to the prognosis of solid tumors such as breast cancer, lung cancer, prostate cancer and colon cancer. The biological activity of each VEGF family member is mediated by one or more of the cell surface VEGF receptor (VEGFR) families, including VEGFR1 (also referred to as FLT-1), VEGFR2 (also referred to as KDR, FLK-1), VEGFR3 (also referred to as FLT-4), etc. where VEGFR1, VEGFR2 is closely related to the generation of blood vessels, the VEGF-C/D/VEGFR3 is closely related to the lymphatic vessel generation. The main biological function of the VEGF family comprises: (1) selectively promoting mitosis of vascular endothelial cells, stimulating endothelial cells proliferation and promoting angiogenesis; (2) increasing the permeability of the blood vessel, particularly the microvascular, so that plasma macromolecules are extravasated and deposited in a matrix outside the blood vessels, and make plasma macromolecules extravasate and deposit in the matrix outside the blood vessels to provide nutrients for the growth of tumor cells and the establishment of new capillary networks (3) promoting the proliferation and metastasis of tumors, the proliferation and metastasis of the tumor depend on the VEGF family to make vascular endothelial cells secrete collagenase and plasminogen to degrade the vascular base membrane, while the newly formed microvascular base membrane inside the tumor tissue is not perfect, which properties allow the tumor to easily enter the blood circulation; and (4) VEGF can be used as an immunosuppressive molecule to inhibit the immune response of the organism, and promote the infiltration and metastasis of malignant tumors (Lasperre-Prost A et al. Immunomodulatory Activity of VEGF in Cancer, Int. Rev. Cell Mol. Biol., 2017; 330: 295-342); (5) Other effects: VEGF family can induce gaps and window openings in epithelial cells, and activate the cytoplasmic vesicles and organelles of epithelial cells; the VEGF family directly stimulates endothelial cells to release proteolytic enzymes, degrade the matrix, release more VEGF family molecules, and accelerate the development of tumors, extracellular proteases can also activate associativity of extracellular matrix and release of VEGF family; VEGF family releases plasma proteins (including fibrinogen) and cellulose network is formed by increasing vascular permeability, which provides a good matrix for tumor growth, development and metastasis; VEGF family promotes the formation of abnormal blood vessels and prevents immune cell invasion and so on.

Clinical studies show the use of anti-VEGF monoclonal antibodies, anti-VEGFR monoclonal antibodies, or soluble VEGFR capable of blocking binding of VEGF families with their receptors and impeding conduction of VEGF family signaling pathways. Bevacizumab (trade name Avastin) developed by Genentech is a recombinant human-mouse chimeric anti-VEGF antibody that can block the binding of VEGF-A and VEGFR, so that VEGFR cannot be activated. This plays an anti-angiogenic effect. Bevacizumab is currently used in the treatment of metastatic colorectal cancer, lung cancer, breast cancer, pancreatic cancer, kidney cancer, etc. Aflibercept developed by Sanofi-aventis and Regeneron is a VEGF-Trap, which is a kind of fusion protein obtained by fusing the second extracellular domain of VEGFR1 and the third extracellular domain of VEGFR2 with the constant region of human IgG1, can exert anti-tumor effect on some tumor patients by inhibiting angiogenesis.

As used herein, the term “specific binding” means that binding to an antigen or target molecule with selectivity and can be distinguished from unwanted or non-specific interaction. The specific binding may be measured by enzyme-linked immunosorbent assay (ELISA) or other techniques familiar to those skilled in the art, such as surface plasmon resonance (SPR) techniques (analyzed on Biacore, et al. Analysis of Agcto-Ciglin Rheumami Using Surface Plasmons Resonance, Glyco. J. 2000, 17, 323-329).

The term “vascular endothelial growth inhibitor domain” refers to a portion of a vascular endothelial growth inhibitor that blocks VEGF/VEGFR signaling conduction, said portion is an domain playing a role in inhibiting vascular endothelial growth. The vascular endothelial growth inhibitor domains may be provided by, for example, a variable domain (also referred to as an antibody variable region) of one or more anti-VEGF antibodies, a variable domain of one or more anti-VEGFR antibodies, or a VEGFR extracellular receptor function.

The term “regulatory T cell” represents a particular T lymphocyte subgroup that is critical to maintaining self-tolerance. The Treg cells with the inhibitor function can be separated from other T lymphocyte by intracellular expression of transcription factor FOXP3 and other cell markers such as CD127^{low}, CTLA-4+, LAP, CD39+, PD-1+, GRP, etc.

“Affinity” or “binding affinity” refers to the inherent binding affinity that reflects interactions between members in binding pair. The affinity of molecule X to its partner Y may generally be represented by a dissociation constant (KD), which is a ratio of dissociation rate constant and associated rate constant (K OFF and K ON, respectively). The affinity may be measured by a common method known in the art. One specific method for measuring affinity is surface plasmon resonance (SPR).

The term “antibody” is used herein in its broadest sense and includes, but is not limited to, monoclonal antibodies, polyclonal antibodies, multispecific antibodies (e.g., bispecific antibodies), provided that they exhibit the desired antigen binding activity. Antibodies can be a complete antibody (e.g., having two full-length light chains and two full-length heavy chains) of any type and subtype (e.g., IgM, IgD, IgG1, IgG2, IgG3, IgG4, IgE, IgA1 and IgA2).

The terms “all-antibody,” “full-length antibody,” “full antibody,” and “complete antibody” are used interchangeably herein to refer to an antibody which has a structure substantially similar to that of a natural antibody.

The term “antibody heavy chain” refers to a greater of two types of polypeptide chains that are present in the antibody molecule in its native presence, which in normal circumstances determine the class to which the antibody belongs.

The term “antibody light chain” refers to a smaller of two types of polypeptide chains present in an antibody molecule in its native presence. κ light chain and λ light chain refers to two major antibody light chain types.

“Bispecific antibody” is an artificial heterozygous antibody having two different heavy chain/light chain pairs and having two different binding sites. Bispecific antibodies can be prepared by a variety of methods, including the attachment of hybridoma fusion or Fab′ fragments.

The term “antigen-binding fragment” of an antibody is a part or a segment of an antibody or antibody chain that has less amino acid residue than a complete or complete antibody or antibody chain, which can bind to an antigen or compete with full antibody (i.e., with a complete antibody derived from an antigen-binding fragment) for binding to an antigen. Antigen binding fragments can be prepared by recombinant DNA techniques, or by enzyme or chemically cleavage of complete antibodies. Antigen binding fragments include, but are not limited to, Fab′, Fab′, F (ab′) 2, Fv′, single chain Fv′. The Fab fragment is a monovalent fragment consisting of VL, VH, Cl and CH1 domains, for example, a Fab fragment can be obtained by digestion of a complete antibody by papain. In addition, F (ab′) 2, which is a dimer of Fab′, is a bivalent fragment by digestion of the complete antibody under the disulfide bond of the hinge region by pepsin. F (ab′) 2 may be reduced under neutral conditions by breaking disulfide bonds in the hinge region, thus converting the F (ab′)₂ dimer to a Fab ‘ monomer. The Fab’ monomer is essentially a Fab fragments with a hinge region (more detailed description of other antibody fragments, please refer to: Fundamental Immunology, edited by W. E. Paul, Raven Press, N.Y. (1993)). The Fv fragment consists of VL and VH domains of the antibody single arm. In addition, although the two domains VL and VH of the Fv fragment are encoded by independent genes, they can be connected through a synthetic linker that enable these two domains to be produced as a single protein chain using recombination methods, and the VL region and VH region in the protein chain are paired to form a single-chain Fv. The antibody fragments can be obtained by chemical methods, recombinant DNA methods or protease digestion methods.

The term “immunoglobulin” refers to a protein having a structure of a naturally occurring antibody. For example, IgG immunoglobulin is a heterotetrameric glycoprotein of about 150,000 Daltons consisting of two light chains and two heavy chains bound by disulfide bonds. From the N-terminal to the C-terminal, each immunoglobulin heavy chain has a variable region (VH), also referred to as a variable heavy chain domain or a heavy chain variable domain, followed by three constant domains (CH1, CH2 and CH3), also referred to as heavy chain constant regions. Similarly, from N-terminal to the C-terminal, each immunoglobulin light chain has a variable region (VL), also referred to as a variable light chain domain or a light chain variable domain, followed by a constant light chain (CL) domain, also referred to as a light chain constant region. The heavy chains of immunoglobulins can belong to one of five categories, called α (IgA), δ (IgD), ε (IgE), γ (IgG) or μ (IgM), some of which can be further divided into sub-classes, such as γ₁ (IgG1), γ₂ (IgG2), γ₃ (IgG₃), γ₄ (IgG₄), α₁ (IgA₁), and α₂ (IgA₂). The light chains of immunoglobulins can be divided into one of two types based on the amino acid sequence of their constant domains, called κ and λ. An immunoglobulin basically consists of two Fab molecules and an Fc domain connected by the hinge region of an immunoglobulin.

The term “Fc domain” or “Fc region” is used herein to define a C-terminal region of an immunoglobulin heavy chain containing at least a portion of the constant region. The term includes a natural sequence Fc region and a variant Fc region. A natural immunoglobulin “Fc domain” includes two or three constant domains, i.e., CH2 domains, CH3 domains, and optional CH4 domains. For example, in a natural antibody, the immunoglobulin Fc domain comprises second and third constant domains (CH2 domains and CH3 domains) derived from two heavy chains of IgG, IgA and IgD antibodies; or second, third, and fourth constant domains (CH2 domains, CH3 domains and CH4 domains) comprising two heavy chains derived from IgM and IgE antibodies. Unless otherwise indicated herein, the number of the amino acid residue in the Fc region or the heavy chain constant region are numbered according to the EU numbering system (also referred to as the EU index) described in Kabat et al. Sequences of Proteins of Immunological Interest, 5th Ed. Public Health Care Service, National Institutes of Health, Bethesda et al. MD. 1991.

“Human immunoglobulin” is an immunoglobulin that possesses an amino acid sequence corresponding to an immunoglobulin produced by a human or human cell or derived from a non-human source utilizing a human immunoglobulin library or other sequences encoding human immunoglobulin.

“Identity Percent (%)” of the amino acid sequence refers to the percentage of amino acid residues in the candidate sequence that are identical to the amino acid residues of the specific amino acid sequence shown in this specification, and without regard to any conservative replacement as part of the sequence identity, after the candidate sequence is compared to the specific amino acid sequence shown in this specification and is introduced in gaps, and if necessary, to reach the maximum sequence identity percent.

The term “effectively linked” means that the specified components are in a relationship that allows them to function in an intended manner.

The term “N-terminal” refers to the last amino acid of the N-terminus, and the term “C-terminal” refers to the last amino acid of the C-terminus.

The term “fusion” refers to two or more components are effectively linked directly by a peptide bond or by one or more peptide linkers.

The term “host cell” refers to a cell into which exogenous polynucleotides have been introduced, including progeny of such cells. Host cells include “transformants” and “transformed cells”, which include primary transformed cells and progeny derived therefrom. Host cells are any type of cell system that can be used to produce the multi-targeting fusion protein of the present invention. Host cells include cultured cells, also including transgenic animals, transgenic plants, or cultured plant tissues or cells inside animal tissues.

The terms “individual” or “subject” are used interchangeably to refer to a mammal. Mammals include, but are not limited to, domesticated animals (e.g., cows, sheep, cats, dogs, and horses), primate (e.g., human and non-human primate such as monkeys), rabbits, and rodents (e.g., mice and rats). In particular, an individuals is human.

The term “treatment” refers to a clinical intervention intended to alter the natural process of a disease in individuals undergoing treatment. The desired therapeutic effect includes, but is not limited to, preventing the occurrence or recurrence of a disease, alleviating symptoms, reducing any direct or indirect pathologies of a disease, preventing metastasis, reducing disease progression rate, ameliorating or alleviating disease states, and alleviating or improving prognosis. In some embodiments, the multi-targeting fusion protein or pharmaceutical composition of the present invention is used to delay disease progression or to slow the progression of disease.

The term “anti-tumor action” refers to a biological effect that can be demonstrated by a variety of means, including but not limited to, for example, reduction in tumor volume, reduced number of tumor cells, reduced tumor cell proliferation, or reduced tumor cell survival. The terms “tumor”, “cancer” and“cancerous disease” are used interchangeably herein to encompass solid tumors and liquid tumors.

### II. Multi-Targeting Fusion Protein

The present invention provides a novel multi-targeting fusion protein comprising (i) a vascular endothelial growth inhibitor domain; (ii) an immunoglobulin Fc domain; and (iii) a CD80 extracellular domain (ECD).

In some embodiments, the (i), (ii) and/or (iii) are optionally effectively linked by a peptide linker.

In some embodiments, the multi-targeting fusion protein of the present invention is a heterotetrameric glycoprotein composed of two fusion protein first subunits and two fusion protein second subunits that are bonded by disulfide bonds.

In some embodiments, the multi-targeting fusion protein of the present invention is homodimer or heterodimer glycoprotein bonded by disulfide bonds.

Multi-targeting fusion proteins of the present invention block vascular endothelial cell growth and activate T lymphocytes. This multi-targeting fusion protein is capable of blocking vascular endothelial cell growth by blocking VEGF/VEGFR pathways, on the other hand, inhibiting PD-1/PD-L1 inhibitory signal pathways by activating CD28/B7 co-stimulatory pathways, inhibiting regulatory T-cell (Treg) functions by the B7/CTLA-4 signaling pathway to activate T lymphocytes, thereby improving the tumor microenvironment and the tumor immunotherapy effect.

In some embodiments, the multi-targeting fusion protein of the present invention binds to VEGF or VEGFR at a dissociation constant (KD) of 10⁻⁸ M or less, e.g., at a dissociation constant (KD) of 10⁻⁹ M to 10⁻¹² M; and specifically binds to CD28, PD-L1, and CTLA-4.

Various components of multi-targeting fusion proteins of the present invention are described below.

Vascular Endothelial Growth Inhibitor Domain

“Vascular endothelial growth inhibitor domains” in multi-targeting fusion proteins of the present invention are capable of specifically binding VEGF and/or VEGFR, including, but not limited to, antigen-binding fragments and/or VEGFR extracellular receptor functional domains derived from anti-VEGF antibodies and/or anti-VEGFR antibodies.

In one embodiment, the multi-targeting fusion protein of the present invention comprises antigen-binding fragments derived from anti-VEGF antibodies and/or anti-VEGFR antibodies so that the multi-targeting fusion protein of the present invention can specifically bind to VEGF and/or VEGFR with high affinity, e.g., at 10⁻⁸ M or less, preferably 10⁻⁹ M to 10⁻¹² M.

Examples of paired heavy chain variable regions (VH) and light chain variable regions (VL) in antigen-binding fragments of anti-VEGF antibodies or anti-VEGFR antibodies included in the multi-targeting fusion proteins of the present invention are provided herein below in Table 1. In some embodiments, the antigen-binding fragment of the multi-targeting fusion protein of the present invention comprise a sequence which is substantially identical to the amino acid sequence of the heavy chain variable region (VH) and/or light chain variable region (VL) of the anti-VEGF antibody and/or anti-VEGFR antibody in Table 1, for example, a sequence having at least 90%, 91%, 92%, 93%, 94%, 95%, 96%, 97%, 98%, 99% or more sequence identity to the paired heavy chain variable region sequence/light chain variable region shown in Table 1.

TABLE 1
Examples of heavy chain variable regions and
light chain variable region sequences in
antigen binding fragments of anti-VEGF
antibodies or anti-VEGFR antibodies.

| | | Sequence |
|---|---|---|
| | | number |
| Variable | Amino acid | (SEQ |
| region | sequence | ID NO:) |
| VH | EVQLVESGGGLVQPGGSLRL | 1 |
| | SCAASGYTFTNYGMNWVRQA | |
| | PGKGLEWVGWINTYTGEPTY | |
| | AADFKRRFTFSLDTSKSTAY | |
| | LQMNSLRAEDTAVYYCAKYP | |
| | HYYGSSHWYFDVWGQG | |
| VL | DIQMTQSPSSLSASVGDRVT | 2 |
| | ITCSASQDISNYLNWYQQKP | |
| | GKAPKVLIYFTSSLHSGVPS | |
| | RFSGSGSGTDFTLTISSLQP | |
| | EDFATYYCQQYSTVPWTFGQ | |
| | G | |
| VH | EVQLVESGGGLVQPGGSLRL | 3 |
| | SCAASGYDFTHYGMNWVRQA | |
| | PGKGLEWVGWINTYTGEPTY | |
| | AADFKRRFTFSLDTSKSTAY | |
| | LQMNSLRAEDTAVYYCAKYP | |
| | YYYGTSHWYFDVWGQG | |
| VL | DIQLTQSPSSLSASVGDRVT | 4 |
| | ITCSASQDISNYLNWYQQKP | |
| | GKAPKVLIYFTSSLHSGVPS | |
| | RFSGSGSGTDFTLTISSLQP | |
| | EDFATYYCQQYSTVPWTFGQ | |
| | G | |
| VH | EVQLVQSGGGLVKPGGSLRL | 5 |
| | SCAASGFTFSSYSMNWVRQA | |
| | PGKGLEWVSSISSSSSYIYY | |
| | ADSVKGRFTISRDNAKNSLY | |
| | LQMNSLRAEDTAVYYCARVT | |
| | DAFDIWGQG | |
| VL | DIQMTQSPSSVSASIGDRVT | 6 |
| | ITCRASQGIDNWLGWYQQKP | |
| | GKAPKLLIYDASNLDTGVPS | |
| | RFSGSGSGTYFTLTISSLQA | |
| | EDFAVYFCQQAKAFPPTFGG | |
| | G | |

In one embodiment, the antigen binding fragment of the multi-targeting fusion protein of the present invention comprises all six heavy chain complementarity determining regions (CDRs) and light chain CDRs contained in paired heavy chain variable region sequences/light chain variable region sequences selected from SEQ ID NO: 1/2, 3/4, and 5/6. Methods and techniques for identifying CDRs in an amino acid sequence of a heavy chain variable region and a light chain variable region are known in the art and may be used to identify CDRs in the amino acid sequence of a particular heavy chain variable region and/or light chain variable region disclosed herein. Exemplary well-known techniques that may be used to identify CDR boundary include, for example, Kabat Definition, Chothia Definition, and ABM Definition. See, for example, Kabat, Sequences of Proteins of Immunological Interest, National Institutes of Health, Bethesda, Md. (1991); Al-Lazikano et al. Standard Modifications for the Canopy Structures of Immunoglobalins. J Mol. Biol. 273: 927-948 (1997); and Martin Ac etal. Modeling of Antibody Hypervariable Loops: a Combined Algorithm, Proc. Natl. Acad. Sci. USA 86: 9268-9272 (1989).

Anti-VEGF antibodies or anti-VEGFR antibodies, which are sources of antigen binding fragments in a multi-targeting fusion protein of the present invention, can be divided into κ type or 2\, type, based on the amino acid sequence of its light chain constant region, preferably κ type.

Examples of amino acid sequences of light chain constant regions of anti-VEGF antibodies or anti-VEGFR antibody are provided herein below.

TABLE 2
Examples of light chain constant
region sequences of antibodies.

| Light | amino acid sequences |
|---|---|
| chain type | of constant region |
| κ | TKVEIKRTVAAPSVFIFPPSDEQ |
| type | LKSGTASVVCLLNNFYPREAKVQ |
| | WKVDNALQSGNSQESVTEQDSKD |
| | STYSLSSTLTLSKADYEKHKVYA |
| | CEVTHQGLSSPVTKSFNRGEC |
| | (SEQ ID NO: 15) |
| λ | TKLTVLGQPKAAPSVTLFPPSSE |
| type | ELQANKATLVCLISDFYPGAVTV |
| | AWKADSSPVKAGVETTTPSKQSN |
| | NKYAASSYLSLTPEQWKSHRSYS |
| | CQVTHEGSTVEKTVAPTECS |
| | (SEQ ID NO: 16) |

The anti-VEGF antibody or anti-VEGFR antibody that is source of the antigen-binding fragment of the multi-targeting fusion protein in the present invention is preferably an IgG antibody, particularly IgG₁ subclass, IgG₂ subclass, IgG₄ subclass, more particularly IgG₄ subclass based on the amino acid sequence of the constant region of the anti-VEGF antibody or anti-VEGFR antibody. Preferably, the IgG4 subclass antibody contains amino acid replacement that prevent arm-exchange at position S228 in the Fc region, particularly amino acid replacement S228P.

Examples of amino acid sequences of antibody heavy chain constant regions are provided herein in Table 3 below.

TABLE 3
Examples of heavy chain constant region
sequences of antibodies.

| Heavy chain | amino acid sequences |
|---|---|
| type | of constant region |
| IgG1 | TLVTVSSASTKGPSVFPLAP |
| | SSKSTSGGTAALGCLVKDYF |
| | PEPVTVSWNSGALTSGVHTF |
| | PAVLQSSGLYSLSSVVTVPS |
| | SSLGTQTYICNVNHKPSNTK |
| | VDKKVEPKSCDKTHTCPPCP |
| | APELLGGPSVFLFPPKPKDT |
| | LMISRTPEVTCVVVDVSHED |
| | PEVKFNWYVDGVEVHNAKTK |
| | PREEQYNSTYRVVSVLTVLH |
| | QDWLNGKEYKCKVSNKALPA |
| | PIEKTISKAKGQPREPQVYT |
| | LPPSREEMTKNQVSLTCLVK |
| | GFYPSDIAVEWESNGQPENN |
| | YKTTPPVLDSDGSFFLYSKL |
| | TVDKSRWQQGNVFSCSVMHE |
| | ALHNHYTQKSLSLSPGK |
| | (SEQ ID NO: 17) |
| IgG2 | TTVTVSTASTKGPSVFPLAP |
| | CSRSTSESTAALGCLVKDYF |
| | PEPVTVSWNSGALTSGVHTF |
| | PAVLQSSGLYSLSSVVTVPS |
| | SNFGTQTYTCNVDHKPSNTK |
| | VDKTVERKCCVECPPCPAPP |
| | VAGPSVFLFPPKPKDTLMIS |
| | RTPEVTCVVVDVSHEDPEVQ |
| | FNWYVDGVEVHNAKTKPREE |
| | QFNSTFRVVSVLTVVHQDWL |
| | NGKEYKCKVSNKGLPAPIEK |
| | TISKTKGQPREPQVYTLPPS |
| | REEMTKNQVSLTCLVKGFYP |
| | SDIAVEWESNGQPENNYKTT |
| | PPMLDSDGSFFLYSKLTVDK |
| | SRWQQGNVFSCSVMHEALHN |
| | HYTQKSLSLSPGK |
| | (SEQ ID NO: 18) |
| IgG4 | TLVTVSSASTKGPSVFPLAP |
| | CSRSTSESTAALGCLVKDYF |
| | PEPVTVSWNSGALTSGVHTF |
| | PAVLQSSGLYSLSSVVTVPS |
| | SSLGTKTYTCNVDHKPSNTK |
| | VDKRVESKYGPPCPPCPAPE |
| | FLGGPSVFLFPPKPKDTLMI |
| | SRTPEVTCVVVDVSQEDPEV |
| | QFNWYVDGVEVHNAKTKPRE |
| | EQFNSTYRVVSVLTVLHQDW |
| | LNGKEYKCKVSNKGLPSSIE |
| | KTISKAKGQPREPQVYTLPP |
| | SQEEMTKNQVSLTCLVKGFY |
| | PSDIAVEWESNGQPENNYKT |
| | TPPVLDSDGSFFLYSRLTVD |
| | KSRWQEGNVFSCSVMHEALH |
| | NHYTQKSLSLSLGK |
| | (SEQ ID NO: 19) |

In one embodiment, the VEGFR extracellular receptor functional region contained in the multi-targeting fusion protein of the present invention is part of or a combination of the extracellular domain of the VEGFR. VEGFR receptor is a tyrosine kinase receptor located on the surface of a cell consisting of 7 immunoglobulin (Ig)-like domains. For example, human VEGFR1 contains 7 Ig-like domains numbered 1, 2, 3, 4, 5, 6, and 7, the Ig-like domain 1 is at the N-terminus of the extracellular domain, and the Ig-like domain 7 is at the C-terminus of the extracellular domain. Unless otherwise indicated herein, the Ig-like domains are numbered sequentially from the N-terminal to the C-terminal of the VEGFR protein. In some embodiments, an VEGFR extracellular receptor functional domain comprises at least one IG-like domain selected from one or more VEGFR of VEGFR1, VEGFR2, and VEGFR3. In some aspects, an VEGFR extracellular receptor functional domain comprises at least 1, 2, 3, 4, 5, 6, but not exceed 7 Ig-like domains of VEGFR. In another aspect, the VEGFR extracellular receptor functional domain comprises 1-7, 1-6, 1-5, 1-4, 1-3, or 1-2 Ig-like domains of VEGFR.

The VEGFR extracellular receptor functional domain that contains at least one Ig-like domain of two or more VEGFRs is also considered herein. In some embodiments, the VEGFR extracellular receptor functional domain comprises at least one Ig-like domain from two or more VEGFR selected from VEGFR1, VEGFR2, and VEGFR3. A VEGFR extracellular receptor functional domain comprises any combination of 7 Ig-like domains of each VEGFR is considered herein. For example, the VEGFR extracellular receptor functional region may comprise an Ig-like domain 2 of VEGFR1 (e.g., human VEGFR1) and an Ig-like domain 3 of VEGFR2 (e.g., human VEGFR2). In another embodiment, the VEGFR extracellular receptor function domain may comprise Ig-like domains 1-3 of VEGFR1 (e.g., human VEGFR1), Ig-like domains 2-3 of VEGFR1 (e.g., human VEGFR1), Ig-like domains 1-3 of VEGFR2 (e.g., human VEGFR2), Ig-like domains 2 of VEGFR1 (e.g., human VEGFR1) and Ig-like domains 3-4 of VEGFR2 (e.g., human VEGFR2), or Ig-like domains 2 of VEGFR1 (e.g., human VEGFR1) and Ig-like domains 3 of VEGFR3 (e.g., human VEGFR3). These Ig-like domains and other Ig-like domains that can be used as part of the VEGFR extracellular receptor functional domain are described more in detail in U.S. Pat. No. 7,531,173; Yu D C et al., Soluble vascular endothelial growth factor decision receptor FP3 exerts potent antiangiogenic effects, Mol. Ther., 2012, 20(3): 938-947 and Holash, J. et al., VEGF-Trap: a VEGF blocker with potent antitumor effects, PNAS, 2002, 99(17): 11393-11398, all documents are hereby incorporated by reference in their entirety. In some embodiments, the VEGFR extracellular receptor functional region has any amino acid sequence selected from the group consisting of amino acid sequences shown in SEQ ID NO: 7-9 in Table 4 or amino acid sequences having at least 90%, 91%, 92%, 93%, 94%, 95%, 96%, 97%, 98%, 99% or more identity to the amino acid sequence shown in SEQ ID NO: 7-9.

TABLE 4
Examples of amino acid sequences of VEGFR
extracellular receptor functional domain
in Multi-Targeting Fusion Protein

| The | |
|---|---|
| VEGFR | |
| extracellular | |
| receptor | |
| functional | |
| domain | Amino acid sequence |
| VEGFR1-D2/V | SDTGRPFVEMYSEIPEIIHM |
| EGFR2-D3 | TEGRELVIPCRVTSPNITVT |
| | LKKFPLDTLIPDGKRIIWDS |
| | RKGFIISNATYKEIGLLTCE |
| | ATVNGHLYKTNYLTHRQTNT |
| | IIDVVLSPSHGIELSVGEKL |
| | VLNCTARTELNVGIDFNWEY |
| | PSSKHQHKKLVNRDLKTQSG |
| | SEMKKFLSTLTIDGVTRSDQ |
| | GLYTCAASSGLMTKKNSTFV |
| | RVHEK |
| | (SEQ ID NO: 7) |
| VEGFR1-D2/V | SDTGRPFVEMYSEIPEIIHM |
| EGFR2-D3-D4 | TEGRELVIPCRVTSPNITVT |
| | LKKFPLDTLIPDGKRIIWDS |
| | RKGFIISNATYKEIGLLTCE |
| | ATVNGHLYKTNYLTHRQTNT |
| | IIDVVLSPSHGIELSVGEKL |
| | VLNCTARTELNVGIDFNWEY |
| | PSSKHQHKKLVNRDLKTQSG |
| | SEMKKFLSTLTIDGVTRSDQ |
| | GLYTCAASSGLMTKKNSTFV |
| | RVHEKPFVAFGSGMESLVEA |
| | TVGERVRIPAKYLGYPPPEI |
| | KWYKNGIPLESNHTIKAGHV |
| | LTIMEVSERDTGNYTVILTN |
| | PISKEKQSHVVSLVVYVPPG |
| | PGD |
| | (SEQ ID NO: 8) |
| VEGFR1-D2 | SDTGRPFVEMYSEIPEIIHM |
| | TEGRELVIPCRVTSPNITVT |
| | LKKFPLDTLIPDGKRIIWDS |
| | RKGFIISNATYKEIGLLTCE |
| | ATVNGHLYKTNYLTHRQTNT |
| | (SEQ ID NO: 9) |

The VEGFR extracellular receptor functional domain of the multi-targeting fusion protein of the present invention can specifically bind to VEGF family with high affinity, e.g., at 10⁻⁸ M or less, preferably 10⁻⁹ M to 10⁻¹² M, and thereby inhibit binding of VEGF family to cell surface VEGFR and subsequent signaling.

Immunoglobulin Fc Domain

The “immunoglobulin Fc domain” in the multi-targeting fusion protein of the present invention includes all the amino acid residues of the naturally-occurring immunoglobulin Fc domain or a part of the amino acid residues of the naturally-occurring immunoglobulin Fc domain. The immunoglobulin Fc domain provides favorable pharmacokinetics properties for the multi-targeting fusion protein of the present invention, including but not limited to long serum half-life. In addition, an immunoglobulin Fc domain also makes it possible to purify the multi-targeting fusion protein of the present invention by, for example, protein A affinity chromatography.

An immunoglobulin Fc domain is usually a dimeric molecule. The immunoglobulin Fc domain can be produced by papain digestion or trypsin digestion of intact (full-length) immunoglobulins or can be recombinantly produced, which comprise a CH2 domain, a CH3 domain and an optional CH4 domain.

In one embodiment, an IgG Fc domain includes an IgG CH2 domain and an IgG CH3 domain. Preferably, the immunoglobulin Fc domain has the amino acid sequence shown in SEQ ID NO: 10-12 in Table 5 or having an amino acid sequence at least 90%, 91%, 92%, 93%, 94%, 95%, 96%, 97%, 98%, 99% or more identical to the amino acid sequence shown in SEQ ID NO: 10-12.

TABLE 5
Examples of amino acid sequence of
immunoglobulin Fc domain in multi-
targeting fusion protein

| Name | Amino acid sequence |
|---|---|
| IgG1 Fc | DKTHTCPPCPAPELLGGPSV |
| | FLFPPKPKDTLMISRTPEVT |
| | CVVVDVSHEDPEVKFNWYVD |
| | GVEVHNAKTKPREEQYNSTY |
| | RVVSVLTVLHQDWLNGKEYK |
| | CKVSNKALPAPIEKTISKAK |
| | GQPREPQVYTLPPSREEMTK |
| | NQVSLTCLVKGFYPSDIAVE |
| | WESNGQPENNYKTTPPVLDS |
| | DGSFFLYSKLTVDKSRWQQG |
| | NVFSCSVMHEALHNHYTQKS |
| | LSLSPGK |
| | (SEQ ID NO: 10) |
| IgG2 Fc | DKTVERKCCVECPPCPAPPV |
| | AGPSVFLFPPKPKDTLMISR |
| | TPEVTCVVVDVSHEDPEVQF |
| | NWYVDGVEVHNAKTKPREEQ |
| | FNSTFRVVSVLTVVHQDWLN |
| | GKEYKCKVSNKGLPAPIEKT |
| | ISKTKGQPREPQVYTLPPSR |
| | EEMTKNQVSLTCLVKGFYPS |
| | DIAVEWESNGQPENNYKTTP |
| | PMLDSDGSFFLYSKLTVDKS |
| | RWQQGNVFSCSVMHEALHNH |
| | YTQKSLSLSPGK |
| | (SEQ ID NO: 11) |
| IgG4 Fc | DKRVESKYGPPCPPCPAPEF |
| | LGGPSVFLFPPKPKDTLMIS |
| | RTPEVTCVVVDVSQEDPEVQ |
| | FNWYVDGVEVHNAKTKPREE |
| | QFNSTYRVVSVLTVLHQDWL |
| | NGKEYKCKVSNKGLPSSIEK |
| | TISKAKGQPREPQVYTLPPS |
| | QEEMTKNQVSLTCLVKGFYP |
| | SDIAVEWESNGQPENNYKTT |
| | PPVLDSDGSFFLYSRLTVDK |
| | SRWQEGNVFSCSVMHEALHN |
| | HYTQKSLSLSLGK |
| | (SEQ ID NO: 12) |

In addition to the sequence defined in SEQ ID NO:10-12, the IgG Fc domain may also include a peptide sequence obtained after additional sequence modification of SEQ ID NO:10-12, for example, a peptide sequence obtained by substituting, deleting or deriving one or more amino acid residues of the amino acid residues in SEQ ID NO:10-12. In one embodiment, the 5228 position in the IgG Fc domain includes amino acid replacement that prevents arm-exchange, especially amino acid replacement S228P. In one embodiment, the IgG4 Fc region contains amino acid replacement S228P.

Extracellular Domain (ECD) of CD 80

The “extracellular domain (ECD) of CD80” in the multi-targeting fusion protein of the present invention comprises all amino acid residues of naturally occurring CD80 ECD or a portion of the amino acid residues comprising naturally occurring CD80 ECD. In some embodiments, the CD80 ECD comprises a CD80 IgV, preferably the CD80 ECD comprises a human CD80 IgV, and more preferably the CD80 ECD has an amino acid sequence as shown in SEQ ID NO: 13 or 14 in Table 6.

TABLE 6
Examples of CD80 ECDVB amino
acid sequencein multi-
targeting fusion protein

| Name | Amino acid sequence |
|---|---|
| CD80-IgV | VIHVTKEVKEVATLSCGHNV |
| | SVEELAQTRIYWQKEKKMVL |
| | TMMSGDMNIWPEYKNRTIFD |
| | ITNNLSIVILALRPSDEGTY |
| | ECVVLKYEKDAFKREHLAEV |
| | TLSVKADFPTPS |
| | (SEQ ID NO: 13) |
| CD80-IgVIgC | VIHVTKEVKEVATLSCGHNV |
| | SVEELAQTRIYWQKEKKMVL |
| | TMMSGDMNIWPEYKNRTIFD |
| | ITNNLSIVILALRPSDEGTY |
| | ECVVLKYEKDAFKREHLAEV |
| | TLSVKADFPTPSISDFEIPT |
| | SNIRRIICSTSGGFPEPHLS |
| | WLENGEELNAINTTVSQDPE |
| | TELYAVSSKLDFNMTTNHSF |
| | MCLIKYGHLRVNQTFNWNTT |
| | KQEHFPDN |
| | (SEQ ID NO: 14) |

In addition to the sequences as defined by SEQ ID NOS: 13 and 14, the CD80 ECD may also contain peptide sequences obtained after additional sequence modifications to SEQ ID NO: 13 and 14, such as peptide sequences obtained after one or more conservative replacements, deletions, or derivatization of amino acid residues in SEQ ID NO: 13 and 14, as long as they have substantially the same activity or function as unmodified peptides. The modified peptide will retain the activity or function associated with the unmodified peptide. The modified peptide typically has an amino acid sequence that is substantially homologous to the amino acid sequence of the unmodified sequence, e.g., an amino acid sequence having at least 90%, 91%, 92%, 93%, 94%, 95%, 96%, 97%, 98%, 99% or more identity to the amino acid sequence shown in SEQ ID NO: 13 or 14.

Peptide Linker

The multi-targeting fusion protein of the present invention will comprise (i) a vascular endothelial growth inhibitor domain; (ii) an immunoglobulin Fc domain; and (iii) a “peptide linker” optionally effectively linked to the CD80 ECD is a peptide of one or more amino acids, typically about 2-20 amino acids. Peptide linkers are known in the art or described herein.

In some embodiments, the peptide linker comprises at least 5 amino acids, preferably comprising the peptide linker selected from AKTTPKLEEGEFSEAR (SEQ ID NO: 20); AKTTPKLEEGEFSEARV (SEQ ID NO: 21); AKTTPKLGG (SEQ ID NO: 22); SAKTTPKLGG (SEQ ID NO: 23); SAKTTP (SEQ ID NO: 24); RADAAP (SEQ ID NO: 25); RADAAPTVS (SEQ ID NO: 26); RADAAAAGGPGS (SEQ ID NO: 27); RADAAAA (SEQ ID NO: 28); SAKTTPKLEEGEFSEARV (SEQ ID NO: 29); ADAAP (SEQ ID NO: 30); DAAPTVSIFPP (SEQ ID NO: 31); TVAAP (SEQ ID NO: 32); TVAAPSVFIFPP (SEQ ID NO: 33); QPKAAP (SEQ ID NO: 34); QPKAAPSVTLFPP (SEQ ID NO: 35); AKTTPP (SEQ ID NO: 36); AKTTPPSVTPLAP (SEQ ID NO: 37); AKTTAP (SEQ ID NO: 38); AKTTAPSVYPLAP (SEQ ID NO: 39); ASTKGP (SEQ ID NO: 40); ASTKGPSVFPLAP (SEQ ID NO: 41); GGGGSGGGGSGGGGS (SEQ ID NO: 42); GENKVEYAPALMALS (SEQ ID NO: 43); GPAKELTPLKEAKVS (SEQ ID NO: 44); GHEAAAVMQVQYPAS (SEQ ID NO: 45); GGGGSGGGGSGGGGSA (SEQ ID NO: 46).

Multi-Targeting Fusion Protein

Provided herein are multi-targeting fusion protein comprising (i) a vascular endothelial growth inhibitor domain; (ii) an immunoglobulin Fc domain; and (iii) a CD80 ECD multi-targeting fusion protein in any order, including, but not limited to, the multi-targeting fusion protein is effectively linked in the order of (I), (II) and (III); the order of (III), (II) and (II); or the order of (III), (II), and (I) from N-terminal to C-terminal.

In one embodiment, the multi-targeting fusion protein comprises a full-length anti-VEGF antibody, a full-length anti-VEGFR antibody, or a full-length anti-VEGF and VEGFR bispecific antibody from the N-terminal to the C-terminal; and one CD80 ECD effectively linked to the C-terminal of each of the two heavy chains of the antibody.

In another embodiment, the multi-targeting fusion protein comprises a full-length anti-VEGF antibody, a full-length anti-VEGFR antibody, or a full-length anti-VEGF and VEGFR bispecific antibody; a CD80 ECD that is operatively linked to the N-terminal of each heavy chain of the two heavy chains of the antibody; and one CD80 ECD that is effectively connected to the N-terminal of each of the two light chains of the antibody.

In yet another embodiment, the multi-targeting fusion protein comprises CD80 ECD from N-terminal to C-terminal; an immunoglobulin Fc domain in dimer form effectively linked at the C-terminal of CD80 ECD; and an antigen-binding fragment derived from an anti-VEGF antibody and/or an anti-VEGFR antibody is effectively linked to the C-terminal of the immunoglobulin Fc domain in dimer form.

In yet another embodiment, the multi-targeting fusion protein comprises CD80 ECD from N-terminal to C-terminal; a dimer-form immunoglobulin Fc domain is effectively linked at C-terminal of CD80 ECD; and a VEGFR extracellular receptor functional domain effectively connected to the C-terminal of the immunoglobulin Fc domain in dimer form.

### III. Production and Purification of the Multi-Targeting Fusion Protein of the Present Invention

The multi-targeting fusion protein of the present invention can be obtained, for example, by solid phase peptide synthesis (for example, Merrifield solid phase synthesis) or recombinant production. The polynucleotides encoding each subunit of the multi-targeting fusion protein are separated and inserted into one or more vectors for further cloning and/or expression in host cells for recombinant production. The polynucleotides can be easily separated and sequenced using conventional methods. In one embodiment, a vector comprising one or more polynucleotides of the present invention is provided, preferably an expression vector.

The expression vector can be constructed using methods well known to those skilled in the art. Expression vectors include but are not limited to viruses, plasmids, cosmids, λ phage or yeast artificial chromosomes (YAC). In a preferred embodiment, a glutamine synthetase high-efficiency expression vector with double expression cassettes is used.

Once an expression vector containing one or more polynucleotides of the present invention has been prepared for expression, the expression vector can be transfected or introduced into a suitable host cell. Various techniques can be used to achieve this purpose, for example, protoplast fusion, calcium phosphate precipitation, electroporation, retroviral transduction, viral transfection, gene gun, liposome-based transfection or other conventional techniques.

In one embodiment, a host cell comprising one or more polynucleotides of the invention is provided. In some embodiments, a host cell comprising an expression vector of the invention is provided. As used herein, the term “host cell” refers to any kind of cellular system that can be engineered to produce the multi-targeting fusion protein of the present invention. Host cells suitable for replicating and supporting the expression of the multi-targeting fusion protein of the present invention are well known in the art. Depending on the needs, such cells can be transfected or transduced with a specific expression vector, and a large number of cells containing the vector can be cultivated for inoculating a large-scale fermenter to obtain a sufficient amount of the multi-targeting fusion protein of the present invention for clinical applications. Suitable host cells include prokaryotic microorganisms, such as Escherichia coli, eukaryotic microorganisms such as filamentous fungi or yeast, or various eukaryotic cells, such as Chinese hamster ovary cells (CHO), insect cells and the like. A mammalian cell line suitable for suspension culture can be used. Examples of useful mammalian host cell lines include SV40 transformed monkey kidney CV1 line (COS-7); human embryonic kidney line (HEK 293 or 293F cells), baby hamster kidney cells (BHK), monkey kidney cells (CV1), African green monkey kidney cells (VERO-76), human cervical cancer cells (HELA), canine kidney cells (MDCK), Buffalo rat liver cells (BRL3A), human lung cells (W138), human liver cells (Hep G2)), CHO cells, NSO cells, myeloma cell lines such as YO, NS0, P3X63 and Sp2/0. For a review of mammalian host cell lines suitable for protein production, see, for example, Yazaki and Wu, Methods in Molecular Biology, Volume 248 (Edited by B.K.C.Lo, Humana Press, Totowa, N.J.), pages 255-268 (2003). In a preferred embodiment, the host cell is a CHO, HEK293 or NSO cell.

Standard techniques for expressing foreign genes in these host cell systems are known in the art. In one embodiment, a method is provided for producing the multi-targeting fusion protein of the present invention, wherein the method comprises culturing the host cell as provided herein under conditions suitable for expressing the multi-targeting fusion protein, the host cell contains a polynucleotide encoding the multi-targeting fusion protein, and the multi-targeting fusion protein is recovered from the host cell (or host cell culture medium).

The multi-targeting fusion protein prepared as described herein can be purified by known existing techniques such as high performance liquid chromatography, ion exchange chromatography, gel electrophoresis, affinity chromatography, size exclusion chromatography and the like. The actual conditions used to purify a particular protein also depend on factors such as net charge, hydrophobicity, hydrophilicity, etc., and these will be obvious to those skilled in the art.

The purity of the multi-targeting fusion protein of the present invention can be determined by any one of a variety of well-known analytical methods, including gel electrophoresis, high-performance liquid chromatography, and the like. The physical/chemical properties and/or biological activity of the multi-targeting fusion protein provided herein can be identified, screened or characterized by various assays known in the art.

### IV. Combination Therapy

PD-1 is an immunosuppressive protein with two ligands, PD-L1 and PD-L2. It is known that the interaction between PD-1 and PD-L1 leads to, for example, a decrease in tumor infiltrating lymphocytes and/or immune evasion of cancer cells. Immunosuppression can be reversed by inhibiting the local interaction of PD-1 with PD-L1 or PD-L2; when the interaction of PD-1 with PD-L2 is also blocked, the effect is additive (Iwai Y. et al., Involvement of PD-L1 on tumor cells in the escape from host immune system and tumor immunotherapy by PD-L1 blockade, Proc. Nat'l. Acad. Sci. USA, 2002, 99: 12293-7). In view of the importance of the signal transduction of the immune checkpoint PD-1 in regulating the immune response, the present invention has developed a pharmaceutical composition for combination therapy, which comprises the multi-targeting fusion protein of the present invention and an anti-PD-1 antibody.

Compared with monotherapy using the multi-targeting fusion protein of the present invention or anti-PD-1 antibody monotherapy, the pharmaceutical composition for combination therapy described herein can provide superior beneficial effects, such as enhanced anti-cancer effects, reduced toxicity and/or reduced side effects. For example, the multi-targeting fusion protein and/or anti-PD-1 antibody of the present invention in the pharmaceutical composition can be administered at a lower dose or shorter administration time required to achieve the same therapeutic effect than monotherapy administration. Therefore, the present invention also discloses the use of a pharmaceutical composition for combination therapy to treat cancer. The effectiveness of the aforementioned pharmaceutical composition can be tested in cell models and animal models known in the art.

The anti-PD-1 antibody contained in the combination therapy can be any anti-PD-1 antibody, as long as it can inhibit or reduce the binding of PD-1 to its ligand, including anti-PD-1 antibodies known in the prior art and anti-PD-1 antibody developed in the future. The anti-PD-1 antibody can specifically bind to PD-1 with high affinity, for example, with a KD of 10⁻⁸M or less, preferably with a KD of 10⁻⁹M to 10⁻¹²M, and thereby block the signal transduction pathway mediated by binding PD-1 to the ligand PD-L1 and/or PD-L2.

Table 7 herein below provides examples of paired heavy chain variable region (VH) and light chain variable region (VL) of the anti-PD-1 antibody included in the combination therapy of the present invention. In some embodiments, the anti-PD-1 antibody in the combination therapy of the present invention comprises a sequence substantially identical to the amino acid sequence shown in Table 7, for example, a sequence having at least 90%, 91%, 92%, 93%, 94%, 95%, 96%, 97%, 98%, 99% or more sequence identity to the paired heavy chain variable region sequence shown in Table 7.

TABLE 7
Examples of sequences of heavy chain variable
region and light chain variable region of
anti-PD-1 antibody in combination therapy

| | | Sequence |
|---|---|---|
| | | number |
| Variable | | (SEQ ID |
| region | Amino acid sequence | NO:) |
| VH | QVQLVESGGGVVQPGRSLRLDC | 47 |
| | KASGITFSNSGMHWVRQAPG | |
| | KGLEWVAVIWYDGSKRYYADSV | |
| | KGRFTISRDNSKNTLFLQMN | |
| | SLRAEDTAVYYCATNDDYWGQG | |
| VL | EIVLTQSPATLSLSPGERATLS | 48 |
| | CRASQSVSSYLAWYQQKPGQAP | |
| | RLLIYDASNRATGIPARFSGSG | |
| | SGTDFTLTISSLEPEDFAVYYC | |
| | QQSSNWPRTFGQG | |
| VH | QVQLVQSGVEVKKPGASVKVSC | 49 |
| | KASGYTFTNYYMYWVRQAP | |
| | GQGLEWMGGINPSNGGTNFNEK | |
| | FKNRVTLTTDSSTTTAYMEL | |
| | KSLQFDDTAVYYCARRDYRFDM | |
| | GFDYWGQG | |
| VL | EIVLTQSPATLSLSPGERATLS | 50 |
| | CRASKGVSTSGYSYLHWYQQKP | |
| | GQAPRLLIYLASYLESGVPARF | |
| | SGSGSGTDFTLTISSLEPEDFA | |
| | VYYCQHSRDLPLTFGGG | |
| VH | QVQLVQSGSELKKPGASVKISC | 51 |
| | KASGYTFTNYGMNWVRQAPG | |
| | QGLQWMGWINTDSGESTYAEEF | |
| | KGRFVFSLDTSVNTAYLQITS | |
| | LTAEDTGMYFCVRVGYDALDYW | |
| | GQG | |
| VL | EIVLTQSPSSLSASVGDRVTIT | 52 |
| | CSARSSVSYMHWFQQKPGK | |
| | APKLWIYRTSNLASGVPSRFSG | |
| | SGSGTSYCLTINSLQPEDFATY | |
| | Y | |
| | CQQRSSFPLTFGGG | |
| VH | EVQLVESGGGLVQPGGSLRLSC | 53 |
| | AASGFTFSSYMMSWVRQAPG | |
| | KGLEWVATISGGGANTYY | |
| | PDSVKGRFTISRDNAKNSLYLQ | |
| | MNSLRAEDTAVYYCARQLYYF | |
| | DYWGQG | |
| VL | DIQMTQSPSSLSASVGDRVTIT | 54 |
| | CLASQTIGTWLTWYQQKPGKAP | |
| | KLLIYTATSLADGVPS | |
| | RFSGSGSGTDFTLTISSLQPED | |
| | FATYYCQQVYSIPWTFGGG | |
| VH | QVTLKESGPALVKPTQTLTLTC | 55 |
| | TFSGFSLSTSGTCVSWIRQPPG | |
| | KALEWLATICWEDSKGYNPSLK | |
| | SRLTISKDTSKNQAVLTMTNMD | |
| | PVDTATYYCARREDSGYFWFPY | |
| | WGQG | |
| VL | NIQMTQSPSSLSASVGDRVTIT | 56 |
| | CKAGQNVNNYLAWYQQKPGK | |
| | APKVLIFNANSLQTGVPSRFSG | |
| | SGSGTDFTLTISSLQPEDFATY | |
| | Y | |
| | CQQYNSWTTFGGG | |
| VH | QVQLQESGPGLVKPSETLSLTC | 57 |
| | TVSGFSLTSYGVHWIRQPPGKG | |
| | LEWLGVIWAGGSTNYNPSLKSR | |
| | LTISKDNSKSQVSLKMSSVTA | |
| | ADTAVYYCARAYGNYWYIDVWG | |
| | QG | |
| VL | DIVMTQSPDSLAVSLGERATIN | 58 |
| | CKASQSVSNDVAWYQQKPGQP | |
| | PKLLINYAFHRFTGVPDRFSGS | |
| | GYGTDFTLTISSLQAEDVAVYY | |
| | CHQAYSSPYTFGGG | |
| VH | VQLVESGGGVVQPGRSLRLDCK | 59 |
| | ASGITFSNYGMHWVRQAPGK | |
| | GLEWVAVIWYDSSRKYYADSVK | |
| | GRFTISRDNSKNTLFLQMNSL | |
| | RAEDTAVYYCATNNDYWGQG | |
| VL | DIQMTQSPSSLSASVGDRVTIT | 60 |
| | CRASQSVSNYLDWYQQKPGKA | |
| | PKLLIYDASTRATGVPSRFSGS | |
| | GSGTDFTLTISSLQPEDFATYY | |
| | CQQNMQLPLTFGQG | |
| VH | DLVQSGAEVKKPGASVKVSCKA | 61 |
| | SGYTFTSYGISWVRQAPGQG | |
| | LEWMGWISAYNGNTNYAQKLQG | |
| | RVTMTTDTSTSTAYMELRSL | |
| | RSDDTAVYYCARGRGYSYGIDA | |
| | FDIWGQG | |
| VL | LSYVLTQPPSVSVSPGQTARIT | 62 |
| | CSGDALPKQYAYWYQQKPGQA | |
| | PVLVIYKDSERPSGIPERFSGS | |
| | SSGTTVTLTISGVQAEDEADYY | |
| | CQSADSSGTYVVFGGG | |
| VH | QGQLVQSGAEVKKPGASVKVSC | 63 |
| | KASGYTFTDYEMHWVRQAP | |
| | GQGLEWMGVIESETGGTAYNQK | |
| | FQGRVTLTADKSSSTAYMEL | |
| | SSLRSEDTAVYYCTREGITTVA | |
| | TTYYWYFDVWGQG | |
| VL | DVVMTQSPLSLPVTLGQPASIS | 64 |
| | CRSSQSIVHSNGNTYLEWYLQK | |
| | PGQSPQLLIYKVSNRFSGVPDR | |
| | FSGSGSGTDFTLKISRVEAEDV | |
| | GVYYCFQGSHVPLTFGQG | |
| VH | EVQLVESGGGLVQPGGSLRLSC | 65 |
| | AASGFTFSSYGMSWVRQAPGK | |
| | GLEWVATISGGGSDTYYADSVK | |
| | GRFTISRDNSKNTLYLQMNSL | |
| | RAEDTAVYYCARQLNYAWFAYW | |
| | GQG | |
| VL | DIVLTQSPASLAVSPGQRATIT | 66 |
| | CRASESVDNYGISFMNWYQQKP | |
| | GQPPKWYTSSNKDTGVPARFSG | |
| | SGSGTDFTLTINPMEAEDTA | |
| | VYYCQQSKEVPWTFGGG | |
| VH | QVQLVQSGAEVKKPGSSVKVSC | 67 |
| | KASGGTFSSYAISWVRQAPGQ | |
| | GLEWMGLIIPMFDTAGYAQKFQ | |
| | GRVAITVDESTSTAYMELSSL | |
| | RSEDTAVYYCARAEHSSTGTFD | |
| | YWGQG | |
| VL | DIQMTQSPSSVSASVGDRVTIT | 68 |
| | CRASQGISSWLAWYQQKPGKA | |
| | PKLLISAASSLQSGVPSRFSGS | |
| | GSGTDFTLTISSLQPEDFATYY | |
| | CQQANHLPFTFGGG | |
| VH | QLQLQESGPGLVKPSETLTLTC | 69 |
| | TVSADSISSTTYYWVWIRQPPG | |
| | KGLEWIGSISYSGSTYYNPSLK | |
| | SRVTVSVDTSKNQFSLKLNSVA | |
| | ATDTALYYCARHLGYNGRYLPF | |
| | DYWGQG | |
| VL | QSALTQPASVSGSPGQSITISC | 70 |
| | TGTSSDVGFYNYVSWYQQHPGK | |
| | APELMIYDVSNRPSGVSDRFSG | |
| | SKSGNTASLTISGLQAEDEADY | |
| | YCSSYTNISTWVFGGG | |
| VH | QVQLVESGGGVVQPGRSLRLTC | 71 |
| | KASGLTFSSSGMHWVRQAPG | |
| | KGLEWVAVIWYDGSKRYYADSV | |
| | KGRFTISRDNSKNTLFLQMN | |
| | SLRAEDTAVYYCATNNDYWGQG | |
| VL | EIVLTQSPATLSLSPGERATLS | 72 |
| | CRASQSVSSYLAWYQQKPGQAP | |
| | RLLIYTASNRATGIPARFSGSG | |
| | SGTDFTLTISSLEPEDFAVYYC | |
| | QQYSNWPRTFGQG | |

In one embodiment, the anti-PD-1 antibody in the combination therapy of the present invention comprises all heavy chain CDRs and light chain CDRs contained in paired heavy chain variable region sequence/light chain variable region sequence selected from SEQ ID NO: 47/48, 49/50, 51/52, 53/54, 55/56, 57/58, 59/60, 61/62, 63/64, 65/66, 67/68, 69/70, and 71/72. Methods and techniques for identifying the CDRs in the amino acid sequence of the heavy chain variable region and the light chain variable region are known in the art, and can be used to identify CDR of the amino acid sequence in the specific heavy chain variable region and/or light chain the variable region disclosed herein. Exemplary well-known techniques that can be used to identify CDR boundary include, for example, the Kabat definition method, the Chothia definition method, and the AbM definition method. See, for example, Kabat, Sequences of Proteins of Immunological Interest, National Institutes of Health, Bethesda, Md. (1991); Al-Lazikano et al. Standard Modifications for the Canopy Structures of Immunoglobalins. J Mol. Biol. 273: 927-948 (1997); and Martin Ac et al. Modeling of Antibody Hypervariable Loops: A combined Algorithm, Proc. Natl. Acad. Sci. USA 86: 9268-9272 (1989).

In one embodiment, the anti-PD-1 antibody in the combination therapy of the present invention is selected from Nivolumab, Pidilizumab and Pembrolizumab.

The pharmaceutical composition of the present invention may contain a “therapeutically effective amount” or a “prophylactically effective amount” of the multi-targeting fusion protein of the present invention and an anti-PD-1 antibody. “Therapeutically effective amount” refers to the amount that is effective to achieve the desired therapeutic result at the required dose and for the required period of time. The therapeutically effective amount can be varied according to various factors such as disease state, age, sex and weight of the individual. A therapeutically effective amount is any amount whose toxic or harmful effect is not as good as the therapeutically beneficial effect. Relative to an untreated subject, a “therapeutically effective amount” preferably inhibits a measurable parameter (such as tumor growth rate) by at least about 20%, more preferably at least about 40%, even more preferably at least about 60%, and still more preferably at least about 80%. The ability of the pharmaceutical composition of the present invention to inhibit a measurable parameter (e.g., tumor volume) can be evaluated in an animal model system that predicts efficacy in human tumors.

“Prophylactically effective amount” refers to an amount that effectively achieves the desired preventive result at the required dose and for the required period of time. Generally, since the prophylactic dose is used in the subject before or in the early stage of the disease, the prophylactically effective amount is less than the therapeutically effective amount.

The multi-targeting fusion protein and anti-PD-1 antibody treatment regimens in the combination therapy of the present invention can be synergistic with each other as demonstrated in the examples in tumor immunity. Therefore, the pharmaceutical composition of the present invention is advantageous for preventing immune escape of tumors. Co-administration of the pharmaceutical composition comprising the multi-targeting fusion protein and anti-PD-1 antibody of the present invention can be implemented by independently administering the multi-targeting fusion protein or anti-PD-1 antibody, or combined preparation of the multi-targeting fusion protein and anti-PD-1 antibody can be administered in a single time. The administration of the multi-targeting fusion protein and anti-PD-1 antibody in the combination therapy of the present invention allows flexibility in dosage and time course.

### V. Use of Multi-Targeting Fusion Protein and Pharmaceutical Composition

The multi-targeting fusion protein and pharmaceutical composition disclosed herein have therapeutic and preventive uses for cancer. For example, the multi-targeting fusion protein and its composition with an anti-PD-1 antibody can be administered to cultured cells in vitro or ex vivo or to a subject, for example, a human subject, to treat and/or prevent multiple kind of cancerous disease.

In one aspect, the present invention relates to the use of a multi-targeting fusion protein or the pharmaceutical composition of the present invention to treat or prevent diseases that require blocking the growth of vascular endothelial cells and activating T cells in a subject, thereby inhibiting or reducing related diseases such as the growth or emergence, metastasis or recurrence of cancerous tumors. Multi-targeting fusion proteins can be used alone to inhibit the growth of cancerous tumors or prevent their appearance. Alternatively, the multi-targeting fusion protein can be administered in combination with other cancer therapeutic/prophylactic agents (e.g., anti-PD-1 antibodies). When the multi-targeting fusion protein of the present invention is administered with an anti-PD-1 antibody, this combination can be administered in any order or simultaneously.

Therefore, in one embodiment, the present invention provides a method of inhibiting the growth of tumor cells in a subject, and the method comprises administering to the subject a therapeutically effective amount of the multi-targeting fusion protein or pharmaceutical composition described herein. In another embodiment, the present invention provides a method for preventing the appearance or metastasis or recurrence of tumor cells in a subject, and the method comprises administering to the subject a prophylactically effective amount of the multi-targeting fusion protein described herein or pharmaceutical composition.

In some embodiments, cancers treated and/or prevented with the multi-targeting fusion protein or pharmaceutical composition include, but are not limited to, solid tumors, hematological cancers (e.g., leukemia, lymphoma, myeloma, e.g., multiple myeloma) and metastatic lesions. In one embodiment, the cancer is a solid tumor. Examples of solid tumors include malignant tumors, for example, sarcomas and cancers of multiple organ systems, such as those that invade the lung, breast, ovary, lymphoid, gastrointestinal (e.g., colon), anus, genitals, and genitourinary tract (e.g., kidney, bladder epithelium, bladder cells, prostate), pharynx, CNS (e.g., brain, neural or glial cells), head and neck, skin (e.g., melanoma), nasopharyngeal (e.g., differentiated or undifferentiated metastatic or locally recurrent nasopharyngeal carcinoma) and those of the pancreas, as well as adenocarcinomas, including malignant tumors such as colon cancer, rectal cancer, renal cell carcinoma, liver cancer, non-small cell lung cancer, small intestine cancer and esophageal cancer. The cancer can be early, middle or late or metastatic cancer.

In some embodiments, the cancer is selected from melanoma, breast cancer, colon cancer, esophageal cancer, gastrointestinal stromal tumor (GIST), kidney cancer (e.g., renal cell carcinoma), liver cancer, non-small cell lung cancer (NSCLC), ovarian cancer, pancreatic cancer, prostate cancer, head and neck tumors, gastric cancer, hematological malignancies (e.g., lymphoma).

The following examples are described to assist the understanding of the present invention. It is not intended and should not be construed in any way to limit the scope of protection of the present invention.

### Examples

### Example 1. Construction of High-Efficiency Glutamine Synthetase Expression Vectors Containing the Target Gene

(1) Synthesis of Nucleotides Encoding Anti-PD1 Antibody BY18.1 and Construction of Expression Vectors

According to the amino acid sequence data of nivolumab numbered 9623 in the International Nonproprietary Name (INN) database, it was optimized to the following nucleotide sequences suitable for expression in Chinese hamster ovarian cancer cells (CHO), and the nucleotide sequences were entrusted to synthesize by Shanghai Jierui Biotechnology Engineering Co., Ltd. The anti-PD1 antibody produced after the expression of the nucleotide sequence is referred to herein as the antibody BY18.1.

The nucleotide sequence (SEQ ID NO: 73) of the light chain (BY18.1L) of the anti-PD1 antibody BY18.1:

CTCGAGGCCACCATGGAGACCGACACACTCCTCCTG
TGGGTGCTGCTGCTGTGGGTGCCTGGCTCCACTGG
CGAGATTGTGCTGACACAGTCCCCCGCTACTCTGA
GCCTGAGCCCTGGCGAGAGGGCTACACTGTCTTGC
AGAGCTTCTCAGTCCGTGTCTTCTTACCTCGCTTG
GTATCAGCAGAAGCCCGGCCAGGCTCCAAGACTGC
TGATCTATGACGCTTCTAACCGCGCTACAGGCATT
CCTGCTAGGTTCAGCGGCAGCGGCTCTGGCACCGA
CTTCACACTCACAATTAGCTCTCTTGAACCTGAGG
ACTTCGCCGTGTACTACTGCCAGCAGTCTAGCAAC
TGGCCTAGAACATTCGGCCAGGGCACTAAGGTGGA
GATTAAGAGAACCGTGGCCGCCCCCAGCGTGTTCA
TCTTCCCTCCCAGCGACGAGCAGCTGAAGTCTGGC
ACCGCCAGCGTGGTGTGCCTGCTGAACAACTTCTA
CCCCCGCGAGGCCAAGGTGCAGTGGAAGGTGGACA
ACGCCCTGCAGAGCGGCAACAGCCAGGAGAGCGTG
ACCGAGCAGGACTCCAAGGACAGCACCTACAGCCT
GAGCAGCACCCTGACCCTGAGCAAGGCCGACTACG
AGAAGCACAAGGTGTACGCCTGCGAGGTGACCCAC
CAGGGACTGTCTAGCCCCGTGACCAAGAGCTTCAA
CCGGGGCGAGTGCTAAGAATTC

The amino acid sequence (SEQ ID NO: 74) of the light chain (BY18.1L) of the anti-PD1 antibody BY18.1:

METDTLLLWVLLLWVPGSTGEIVLTQSPATLSLSPGERATLSCRASQSVS
SYLAWYQQKPGQAPRLLIYDASNRATGIPARFSGSGSGTDFTLTISSLEP
EDFAVYYCQQSSNWPRTFGQGTKVEIKRTVAAPSVFIFPPSDEQLKSGTA
SVVCLLNNFYPREAKVQWKVDNALQSGNSQESVTEQDSKDSTYSLSSTLT
LSKADYEKHKVYACEVTHQGLSSPVTKSFNRGEC

The nucleotide sequence (SEQ ID NO: 75) of the heavy chain (BY18.1H) of the anti-PD1 antibody BY18.1:

TCTAGAGCCACCATGGAGACCGACACCCTGCTGCTGTGGGTGCTGCTCCT
GTGGGTGCCTGGCTCCACAGGCCAGGTGCAGCTCGTGGAGTCCGGCGGCG
GCGTGGTGCAGCCCGGCAGATCCCTCAGACTGGACTGCAAGGCATCCGGC
ATTACATTCTCTAACTCTGGAATGCACTGGGTGAGACAGGCTCCTGGCAA
GGGCCTGGAATGGGTGGCCGTGATTTGGTACGACGGCTCTAAGAGATACT
ACGCTGACTCCGTGAAGGGCCGGTTCACAATTAGCAGAGACAACTCCAAG
AACACTCTGTTCCTCCAGATGAACAGCCTGAGAGCCGAGGACACCGCTGT
GTACTACTGCGCCACCAACGACGACTACTGGGGCCAGGGCACCCTCGTGA
CAGTGTCTTCCGCCTCCACCAAGGGCCCTTCCGTGTTCCCTCTGGCCCCT
TGCTCCCGCTCCACCTCCGAGTCCACCGCCGCCCTGGGCTGCCTGGTGAA
GGACTACTTCCCTGAGCCTGTGACCGTGTCCTGGAACTCCGGCGCCCTGA
CCTCCGGCGTGCACACCTTCCCTGCCGTGCTGCAGTCCTCCGGCCTGTAC
TCCCTGTCCTCCGTGGTGACCGTGCCTTCCTCCTCCCTGGGCACCAAGAC
CTACACCTGCAACGTGGACCACAAGCCTTCCAACACCAAGGTGGACAAGC
GCGTGGAGTCCAAGTACGGCCCTCCTTGCCCTCCTTGCCCTGCCCCTGAG
TTCCTGGGCGGCCCTTCCGTGTTCCTGTTCCCTCCTAAGCCTAAGGACAC
CCTGATGATCTCCCGCACCCCTGAGGTGACCTGCGTGGTGGTGGACGTGT
CCCAGGAGGACCCTGAGGTGCAGTTCAACTGGTACGTGGACGGCGTGGAG
GTGCACAACGCCAAGACCAAGCCTCGCGAGGAGCAGTTCAACTCCACCTA
CCGCGTGGTGTCCGTGCTGACCGTGCTGCACCAGGACTGGCTGAACGGCA
AGGAGTACAAGTGCAAGGTGTCCAACAAGGGCCTGCCTTCCTCCATCGAG
AAGACCATCTCCAAGGCCAAGGGCCAGCCTCGCGAGCCTCAGGTGTACAC
CCTGCCTCCTTCCCAGGAGGAGATGACCAAGAACCAGGTGTCCCTGACCT
GCCTGGTGAAGGGCTTCTACCCTTCCGACATCGCCGTGGAGTGGGAGTCC
AACGGCCAGCCTGAGAACAACTACAAGACCACCCCTCCTGTGCTGGACTC
CGACGGCTCCTTCTTCCTGTACTCCCGCCTGACCGTGGACAAGTCCCGCT
GGCAGGAGGGCAACGTGTTCTCCTGCTCCGTGATGCACGAGGCCCTGCAC
AACCACTACACCCAGAAGTCCCTGTCCCTGTCCCTGGGCAAGTAAGTCGA
C

The heavy chain (BY18.1H) amino acid sequence (SEQ ID NO: 76) of the anti-PD1 antibody BY18.1:

METDTLLLWVLLLWVPGSTGQVQLVESGGGVVQPGRSLRLDCKASGITFS
NSGMHWVRQAPGKGLEWVAVIWYDGSKRYYADSVKGRFTISRDNSKNTLF
LQMNSLRAEDTAVYYCATNDDYWGQGTLVTVSSASTKGPSVFPLAPCSRS
TSESTAALGCLVKDYFPEPVTVSWNSGALTSGVHTFPAVLQSSGLYSLSS
VVTVPSSSLGTKTYTCNVDHKPSNTKVDKRVESKYGPPCPPCPAPEFLGG
PSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYT
QKSLSLSLGK

Wherein the underlined part “METDTLLLWVLLLWVPGSTG” is a signal peptide sequence.

The above nucleotide sequences encoding BY18.1L and nucleotide sequences encoding BY18.1H were synthesized by Shanghai Jierui Bioengineering Co., Ltd. The nucleotides encoding BY18.1L were respectively digested with XhoI-EcoRI double enzymes, and the high-efficiency glutamine synthetase expression vectors (Patent No.: CN104195173B, obtained from Beijing Biyang Biotechnology Co., Ltd.) were digested with XhoI-EcoRI double enzymes, and then nucleotides encoding BY18.1L digested by XhoI-EcoRI double enzymes were linked by ligase to glutamine synthetase high-efficiency expression vectors having double expression cassettes digested by XhoI-EcoRI double enzymes, and the high-efficiency glutamine synthetase expression vectors having double expression cassettes which have been introduced into nucleotide encoding BY18.1L were obtained; and then, the nucleotides encoding BY18.1H were respectively digested with XbaI-SalI double enzymes, and the high-efficiency glutamine synthetase expression vectors were digested with XbaI-SalI double enzymes, and then nucleotides encoding BY18.1H digested by XbaI-SalI double enzymes were linked by ligase to glutamine synthetase high-efficiency expression vectors having double expression cassettes digested by XbaI-SalI double enzymes, thereby the high-efficiency glutamine synthetase expression vectors having double expression cassettes which have been introduced into nucleotides encoding BY18.1H were obtained, after the correct expression verified by sequencing the anti-PD1 antibodies BY18.1 were obtained.

Alternatively, nucleotides encoding BY18.1L can also be ligated into high-efficiency glutamine synthetase high-efficiency expression vectors with double expression cassette which have been introduced into nucleotides encoding BY18.1H, and the antibodies BY18.1 were expressed and obtained.

(2) Synthesis of Nucleotides Encoding Fusion Protein Containing CD80 Extracellular Domain and Immunoglobulin Fc Region and Construction of Expression Vectors

According to the sequence of the CD80 extracellular domain in Table 1, and the IgG4Fc sequence in Table 6 were optimized to nucleotide sequences suitable for expression in Chinese Hamster Ovarian Cancer Cells (CHO), and polynucleotide sequences SEQ ID NO: 77 as follows were entrusted to synthesize by Shanghai Jierui Bioengineering Co., Ltd. The CD80-Fc fusion protein produced after the expression of the nucleotide sequence is also referred to herein as the fusion protein BY31.19.

Nucleotide sequence of the fusion protein BY31.19 (CD80-Fc, IgG4) (SEQ ID NO: 77)

CTCGAGGCCACCATGGAGACCGACACACTCCTCCTGTGGGTGCTGCTGCT
GTGGGTGCCTGGCTCCACTGGCGTCATCCATGTGACCAAAGAGGTCAAAG
AAGTCGCAACTCTGTCTTGCGGACACAACGTCTCCGTCGAAGAACTCGCC
CAGACTAGAATATATTGGCAGAAGGAAAAGAAGATGGTGCTCACCATGAT
GTCCGGCGATATGAACATTTGGCCTGAGTACAAGAACCGGACAATCTTCG
ATATTACTAATAACCTGAGTATCGTGATTCTCGCTCTGCGCCCTAGCGAC
GAGGGCACATACGAGTGTGTGGTGCTGAAGTACGAGAAGGATGCATTCAA
GCGGGAGCACCTCGCAGAGGTGACACTCTCCGTGAAGGCCGACTTCCCAA
CCCCATCTGACAAGCGCGTGGAGTCCAAGTACGGCCCTCCTTGCCCTCCT
TGCCCTGCCCCTGAGTTCCTGGGCGGCCCTTCCGTGTTCCTGTTCCCTCC
TAAGCCTAAGGACACCCTGATGATCTCCCGCACCCCTGAGGTGACCTGCG
TGGTGGTGGACGTGTCCCAGGAGGACCCTGAGGTGCAGTTCAACTGGTAC
GTGGACGGCGTGGAGGTGCACAACGCCAAGACCAAGCCTCGCGAGGAGCA
GTTCAACTCCACCTACCGCGTGGTGTCCGTGCTGACCGTGCTGCACCAGG
ACTGGCTGAACGGCAAGGAGTACAAGTGCAAGGTGTCCAACAAGGGCCTG
CCTTCCTCCATCGAGAAGACCATCTCCAAGGCCAAGGGCCAGCCTCGCGA
GCCTCAGGTGTACACCCTGCCTCCTTCCCAGGAGGAGATGACCAAGAACC
AGGTGTCCCTGACCTGCCTGGTGAAGGGCTTCTACCCTTCCGACATCGCC
GTGGAGTGGGAGTCCAACGGCCAGCCTGAGAACAACTACAAGACCACCCC
TCCTGTGCTGGACTCCGACGGCTCCTTCTTCCTGTACTCCCGCCTGACCG
TGGACAAGTCCCGCTGGCAGGAGGGCAACGTGTTCTCCTGCTCCGTGATG
CACGAGGCCCTGCACAACCACTACACCCAGAAGTCCCTGTCCCTGTCCCT
GGGCTAAGAATTC

The amino acid sequence of the fusion protein BY31.19 (CD80-Fc, IgG4) (SEQ ID NO: 78)

METDTLLLWVLLLWVPGSTGVIHVTKEVKEVATLSCGHNVSVEELAQTRI
YWQKEKKMVLTMMSGDMNIWPEYKNRTIFDITNNLSIVILALRPSDEGTY
ECVVLKYEKDAFKREHLAEVTLSVKADFPTPSDKRVESKYGPPCPPCPAP
EFLGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEAL
HNHYTQKSLSLSLG

Wherein the amino acid sequence “METDTLLLWVLLLWVPGSTG” is a signal peptide.

Using the same method as in Example 1(1) above, the nucleotides encoding BY31.19 digested by XhoI-EcoRI double enzymes were linked to glutamine synthetase high-efficiency expression vectors with double expression cassettes (Patent No.: CN104195173B, obtained From Beijing Biyang Biotechnology Co., Ltd.). The recombinant vectors were sequenced to verify that it was correct and used for CD80-Fc fusion protein expression. The expressed CD80-Fc fusion protein was named fusion protein BY31.19.

(3) Synthesis of Nucleotides Encoding Fusion Protein BY24.4 Containing Anti-VEGF Antibody and CD80 Extracellular Domain and Construction of Expression Vectors

According to the sequence of the CD80 extracellular domain in Table 1, the heavy chain variable region and light chain variable region sequence of the anti-VEGF antibody in Table 2, the heavy chain constant region sequence of the antibody in Table 3, and the constant region sequence of the light chain of the antibody in Table 4, and the peptide linker sequence of SEQ ID NO: 20-46 were optimized to nucleotide sequences suitable for expression in Chinese hamster ovarian cancer cells (CHO), and Shanghai Jierui Bioengineering Co., Ltd. was entrusted to synthesize the following polynucleotide sequence shown in SEQ ID NO: 79 and 81. The anti-VEGF antibody-CD80 fusion protein produced after the expression of the nucleotide sequence is referred to herein as the fusion protein BY24.4.

The nucleotide sequence of the first subunit (BY24.4L) of the fusion protein BY24.4 (κ, IgG4) (SEQ ID NO: 79)

CTCGAGGCCACCATGGAGACAGATACTCTGCTGCTCTGGGTCCTGCTGCT
GTGGGTGCCAGGCAGCACAGGCGACATCCAGATGACCCAGTCCCCTTCCT
CCCTGTCTGCCTCCGTGGGCGACAGGGTGACCATCACCTGTTCCGCATCC
CAGGATATCTCCAACTACCTGAACTGGTATCAGCAGAAGCCAGGAAAGGC
CCCCAAGGTGCTGATCTACTTTACTTCTTCCCTGCATTCCGGCGTGCCCT
CCAGGTTCTCTGGCTCCGGGTCTGGAACCGACTTTACACTGACAATCTCC
TCTCTGCAGCCAGAAGACTTTGCCACCTATTACTGCCAGCAGTACTCCAC
TGTCCCCTGGACCTTCGGGCAAGGAACCAAAGTGGAAATCAAGAGGACCG
TGGCCGCCCCCAGCGTGTTCATCTTCCCTCCCAGCGACGAGCAGCTGAAG
TCTGGCACCGCCAgCGTGGTGTGCCTGCTGAACAACTTCTACCCCCGCGA
GGCCAAGGTGCAGTGGAAGGTGGACAACGCCCTGCAgAGCGGCAACAGCC
AGGAGAGCGTGACCGAGCAGGACTCCAAGGACAGCACCTACAGCCTGAGC
AGCACCCTGACCCTGAGCAAGGCCGACTACGAGAAGCACAAGGTGTACGC
CTGCGAGGTGACCCACCAGGGACTGTCTAGCCCCGTGACCAAGAGCTTCA
ACCGGGGCGAGTGCTAAGAATTC

The amino acid sequence of the first subunit (BY24.4L) of the fusion protein BY24.4 (κ, IgG4) (SEQ ID NO: 80)

METDTLLLWVLLLWVPGSTGDIQMTQSPSSLSASVGDRVTITCSASQDIS
NYLNWYQQKPGKAPKVLIYFTSSLHSGVPSRFSGSGSGTDFTLTISSLQP
EDFATYYCQQYSTVPWTFGQGTKVEIKRTVAAPSVFIFPPSDEQLKSGTA
SVVCLLNNFYPREAKVQWKVDNALQSGNSQESVTEQDSKDSTYSLSSTLT
LSKADYEKHKVYACEVTHQGLSSPVTKSFNRGEC

The nucleotide sequence of the second subunit (BY24.4H) of the fusion protein BY24.4 (κ, IgG4) (SEQ ID NO: 81)

TCTAGAGCCACCATGGAAACCGACACACTGCTGCTGTGGGTCCTGCTGCT
GTGGGTGCCAGGCTCTACAGGCGAAGTGCAGCTGGTCGAATCTGGCGGCG
GACTGGTGCAACCAGGGGGCAGCCTGAGGCTGTCCTGCGCCGCTTCTGGA
TACACCTTTACCAATTACGGCATGAATTGGGTGAGGCAGGCTCCTGGCAA
GGGCCTGGAGTGGGTGGGATGGATCAACACCTACACCGGCGAGCCCACCT
ACGCCGCTGACTTTAAGAGGAGGTTCACCTTCTCTCTGGACACCTCCAAG
TCCACCGCTTACCTGCAAATGAACTCTCTGAGGGCTGAAGACACCGCCGT
GTACTACTGCGCCAAGTACCCCCATTACTACGGGTCCTCCCACTGGTACT
TCGACGTGTGGGGCCAGGGCACCCTCGTGACAGTGTCTTCCGCCTCCACC
AAGGGCCCTTCCGTGTTCCCTCTGGCCCCTTGCTCCCGCTCCACCTCCGA
GTCCACCGCCGCCCTGGGCTGCCTGGTGAAGGACTACTTCCCTGAGCCTG
TGACCGTGTCCTGGAACTCCGGCGCCCTGACCTCCGGCGTGCACACCTTC
CCTGCCGTGCTGCAGTCCTCCGGCCTGTACTCCCTGTCCTCCGTGGTGAC
CGTGCCTTCCTCCTCCCTGGGCACCAAGACCTACACCTGCAACGTGGACC
ACAAGCCTTCCAACACCAAGGTGGACAAGCGCGTGGAGTCCAAGTACGGC
CCTCCTTGCCCTCCTTGCCCTGCCCCTGAGTTCCTGGGCGGCCCTTCCGT
GTTCCTGTTCCCTCCTAAGCCTAAGGACACCCTGATGATCTCCCGCACCC
CTGAGGTGACCTGCGTGGTGGTGGACGTGTCCCAGGAGGACCCTGAGGTG
CAGTTCAACTGGTACGTGGACGGCGTGGAGGTGCACAACGCCAAGACCAA
GCCTCGCGAGGAGCAGTTCAACTCCACCTACCGCGTGGTGTCCGTGCTGA
CCGTGCTGCACCAGGACTGGCTGAACGGCAAGGAGTACAAGTGCAAGGTG
TCCAACAAGGGCCTGCCTTCCTCCATCGAGAAGACCATCTCCAAGGCCAA
GGGCCAGCCTCGCGAGCCTCAGGTGTACACCCTGCCTCCTTCCCAGGAGG
AGATGACCAAGAACCAGGTGTCCCTGACCTGCCTGGTGAAGGGCTTCTAC
CCTTCCGACATCGCCGTGGAGTGGGAGTCCAACGGCCAGCCTGAGAACAA
CTACAAGACCACCCCTCCTGTGCTGGACTCCGACGGCTCCTTCTTCCTGT
ACTCCCGCCTGACCGTGGACAAGTCCCGCTGGCAGGAGGGCAACGTGTTC
TCCTGCTCCGTGATGCACGAGGCCCTGCACAACCACTACACCCAGAAGTC
CCTGTCCCTGTCCCTGGGCGGCGGAGGATCTGGCGGCGGAGGCAGTGGAG
GCGGCGGAAGTGTGATCCATGTAACAAAAGAAGTGAAGGAAGTGGCTACA
CTCTCTTGCGGCCACAACGTGTCCGTGGAGGAACTAGCTCAGACCCGGAT
CTATTGGCAGAAAGAAAAGAAGATGGTGCTGACCATGATGTCCGGCGACA
TGAACATTTGGCCAGAGTACAAGAACCGCACAATTTTCGACATTACAAAC
AACCTCTCTATTGTGATTCTGGCTCTCAGGCCTAGCGACGAGGGCACATA
CGAGTGCGTGGTGCTCAAGTACGAGAAGGACGCTTTCAAGCGGGAGCACC
TCGCTGAGGTGACCCTGTCCGTGAAGGCCGACTTCCCTACTCCATCTTAA
GTCGAC

The amino acid sequence of the second subunit (BY24.4H) of the fusion protein BY24.4 (κ, IgG4) (SEQ ID NO: 82)

METDTLLLWVLLLWVPGSTGEVQLVESGGGLVQPGGSLRLSCAASGYTFT
NYGMNWVRQAPGKGLEWVGWINTYTGEPTYAADFKRRFTFSLDTSKSTAY
LQMNSLRAEDTAVYYCAKYPHYYGSSHWYFDVWGQGTLVTVSSASTKGPS
VFPLAPCSRSTSESTAALGCLVKDYFPEPVTVSWNSGALTSGVHTFPAVL
QSSGLYSLSSVVTVPSSSLGTKTYTCNVDHKPSNTKVDKRVESKYGPPCP
PCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSV
MHEALHNHYTQKSLSLSLGGGGSGGGGSGGGGSVIHVTKEVKEVATLSCG
HNVSVEELAQTRIYWQKEKKMVLTMMSGDMNIWPEYKNRTIFDITNNLSI
VILALRPSDEGTYECVVLKYEKDAFKREHLAEVTLSVKADFPTPS

Wherein the amino acid sequence “METDTLLLWVLLLWVPGSTG” is a signal peptide.

Using the same method as in Example 1 (1) above, the nucleotides encoding BY24.4L digested by XhoI-EcoRI double enzymes were linked to glutamine synthetase high-efficiency expression vectors with double expression cassette (patent No.: CN104195173B, obtained From Beijing Biyang Biotechnology Co., Ltd.); And then, the nucleotides encoding BY24.4H digested by XbaI-SalI double enzymes were linked to glutamine synthetase high-efficiency expression vectors connected to nucleotides encoding BY24.4L with a double expression cassette; or vice versa. The recombinant vectors were sequenced and verified to be correct then used for expression. The expressed anti-VEGF antibody-CD80 fusion protein was named fusion protein BY24.4.

(4) Synthesis of Nucleotides Encoding Fusion Protein BY24.5 Containing Anti-VEGFR Antibody and CD80 Extracellular Domain and Construction of Expression Vectors

According to the sequence of the CD80 extracellular domain in Table 1, the heavy chain variable region and light chain variable region sequence of the anti-VEGFR antibody in Table 2, the heavy chain constant region sequence of the antibody in Table 3, the constant region sequence of the light chain of the antibody in Table 4, and the peptide linker sequence of SEQ ID NO: 20-46 were optimized to nucleotide sequences suitable for expression in Chinese hamster ovarian cancer cells (CHO), and Shanghai Jierui Bioengineering Co., Ltd. was entrusted to synthesize the following polynucleotide sequences shown in SEQ ID NO: 83 and 85. The anti-VEGFR antibody-CD80 fusion protein produced after the expression of the nucleotide sequence is referred to herein as the fusion protein BY24.5.

The nucleotide sequence of the first subunit (BY24.5L) of the fusion protein BY24.5 (κ, IgG4) (SEQ ID NO: 83)

CTCGAGGCCACCATGGAGACAGATACTCTGCTGCTCTGGGTCCTGCTGCT
GTGGGTGCCAGGCAGCACAGGCGACATCCAGATGACCCAGTCTCCTAGCT
CCGTGAGCGCCTCTATCGGCGACAGAGTGACCATCACATGCAGAGCTTCT
CAGGGCATCGACAACTGGCTCGGCTGGTACCAGCAGAAGCCTGGCAAGGC
CCCAAAGCTGCTGATCTACGACGCCTCTAACCTGGACACCGGCGTGCCAT
CCCGGTTCAGCGGCTCTGGCTCCGGCACATACTTCACACTCACAATCTCT
TCCCTGCAGGCCGAGGACTTCGCTGTGTACTTCTGCCAGCAGGCTAAGGC
TTTCCCACCTACATTCGGCGGCGGAACCAAAGTGGAAATCAAGAGGACCG
TGGCCGCCCCCAGCGTGTTCATCTTCCCTCCCAGCGACGAGCAGCTGAAG
TCTGGCACCGCCAgCGTGGTGTGCCTGCTGAACAACTTCTACCCCCGCGA
GGCCAAGGTGCAGTGGAAGGTGGACAACGCCCTGCAgAGCGGCAACAGCC
AGGAGAGCGTGACCGAGCAGGACTCCAAGGACAGCACCTACAGCCTGAGC
AGCACCCTGACCCTGAGCAAGGCCGACTACGAGAAGCACAAGGTGTACGC
CTGCGAGGTGACCCACCAGGGACTGTCTAGCCCCGTGACCAAGAGCTTCA
ACCGGGGCGAGTGCTAAGAATTC

The amino acid sequence of the first subunit (BY24.5L) of the fusion protein BY24.5 (κ, IgG4) (SEQ ID NO: 84)

METDTLLLWVLLLWVPGSTGDIQMTQSPSSVSASIGDRVTITCRASQGID
NWLGWYQQKPGKAPKLLIYDASNLDTGVPSRFSGSGSGTYFTLTISSLQA
EDFAVYFCQQAKAFPPTFGGGTKVEIKRTVAAPSVFIFPPSDEQLKSGTA
SVVCLLNNFYPREAKVQWKVDNALQSGNSQESVTEQDSKDSTYSLSSTLT
LSKADYEKHKVYACEVTHQGLSSPVTKSFNRGEC

The nucleotide sequence of the second subunit (BY24.5H) of the fusion protein BY24.5 (κ, IgG4) (SEQ ID NO: 85)

TCTAGAGCCACCATGGAAACCGACACACTGCTGCTGTGGGTCCTGCTGCT
GTGGGTGCCAGGCTCTACAGGCGAGGTGCAGCTCGTGCAGTCTGGCGGCG
GCCTCGTGAAGCCAGGCGGCTCCCTCAGACTGTCTTGCGCTGCTAGCGGC
TTCACATTCTCTTCCTACTCTATGAACTGGGTGAGACAGGCTCCCGGCAA
GGGCCTGGAGTGGGTGTCCAGCATCTCTTCCTCTAGCTCTTACATCTACT
ACGCCGACTCCGTGAAGGGCAGATTCACAATCAGCCGGGACAACGCCAAG
AACTCCCTGTACCTGCAGATGAACAGCCTGAGAGCCGAGGACACCGCTGT
GTACTACTGCGCTAGAGTGACCGACGCTTTCGACATCTGGGGCCAGGGCA
CTCTCGTGACAGTGTCTTCCGCCTCCACCAAGGGCCCTTCCGTGTTCCCT
CTGGCCCCTTGCTCCCGCTCCACCTCCGAGTCCACCGCCGCCCTGGGCTG
CCTGGTGAAGGACTACTTCCCTGAGCCTGTGACCGTGTCCTGGAACTCCG
GCGCCCTGACCTCCGGCGTGCACACCTTCCCTGCCGTGCTGCAGTCCTCC
GGCCTGTACTCCCTGTCCTCCGTGGTGACCGTGCCTTCCTCCTCCCTGGG
CACCAAGACCTACACCTGCAACGTGGACCACAAGCCTTCCAACACCAAGG
TGGACAAGCGCGTGGAGTCCAAGTACGGCCCTCCTTGCCCTCCTTGCCCT
GCCCCTGAGTTCCTGGGCGGCCCTTCCGTGTTCCTGTTCCCTCCTAAGCC
TAAGGACACCCTGATGATCTCCCGCACCCCTGAGGTGACCTGCGTGGTGG
TGGACGTGTCCCAGGAGGACCCTGAGGTGCAGTTCAACTGGTACGTGGAC
GGCGTGGAGGTGCACAACGCCAAGACCAAGCCTCGCGAGGAGCAGTTCAA
CTCCACCTACCGCGTGGTGTCCGTGCTGACCGTGCTGCACCAGGACTGGC
TGAACGGCAAGGAGTACAAGTGCAAGGTGTCCAACAAGGGCCTGCCTTCC
TCCATCGAGAAGACCATCTCCAAGGCCAAGGGCCAGCCTCGCGAGCCTCA
GGTGTACACCCTGCCTCCTTCCCAGGAGGAGATGACCAAGAACCAGGTGT
CCCTGACCTGCCTGGTGAAGGGCTTCTACCCTTCCGACATCGCCGTGGAG
TGGGAGTCCAACGGCCAGCCTGAGAACAACTACAAGACCACCCCTCCTGT
GCTGGACTCCGACGGCTCCTTCTTCCTGTACTCCCGCCTGACCGTGGACA
AGTCCCGCTGGCAGGAGGGCAACGTGTTCTCCTGCTCCGTGATGCACGAG
GCCCTGCACAACCACTACACCCAGAAGTCCCTGTCCCTGTCCCTGGGCGG
CGGAGGATCTGGCGGCGGAGGCAGTGGAGGCGGCGGAAGTGTGATCCATG
TAACAAAAGAAGTGAAGGAAGTGGCTACACTCTCTTGCGGCCACAACGTG
TCCGTGGAGGAACTAGCTCAGACCCGGATCTATTGGCAGAAAGAAAAGAA
GATGGTGCTGACCATGATGTCCGGCGACATGAACATTTGGCCAGAGTACA
AGAACCGCACAATTTTCGACATTACAAACAACCTCTCTATTGTGATTCTG
GCTCTCAGGCCTAGCGACGAGGGCACATACGAGTGCGTGGTGCTCAAGTA
CGAGAAGGACGCTTTCAAGCGGGAGCACCTCGCTGAGGTGACCCTGTCCG
TGAAGGCCGACTTCCCTACTCCATCTTAAGTCGAC

The amino acid sequence of the second subunit (BY24.5H) of the fusion protein BY24.5 (κ, IgG4) (SEQ ID NO: 86)

METDTLLLWVLLLWVPGSTGEVQLVQSGGGLVKPGGSLRLSCAASGFTFS
SYSMNWVRQAPGKGLEWVSSISSSSSYIYYADSVKGRFTISRDNAKNSLY
LQMNSLRAEDTAVYYCARVTDAFDIWGQGTLVTVSSASTKGPSVFPLAPC
SRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEALHN
HYTQKSLSLSLGGGGSGGGGSGGGGSVIHVTKEVKEVATLSCGHNVSVEE
LAQTRIYWQKEKKMVLTMMSGDMNIWPEYKNRTIFDITNNLSIVILALRP
SDEGTYECVVLKYEKDAFKREHLAEVTLSVKADFPTPS

Wherein the amino acid sequence “METDTLLLWVLLLWVPGSTG” is a signal peptide.

Using the same method as in Example 1 (1) above, the nucleotides encoding BY24.5L digested by XhoI-EcoRI double enzymes were linked to glutamine synthetase high-efficiency expression vectors with double expression cassette (patent No.: CN104195173B, obtained From Beijing Biyang Biotechnology Co., Ltd.); And then, the nucleotides encoding BY24.5H digested by XbaI-SalI double enzymes were linked to glutamine synthetase high-efficiency expression vectors connected to nucleotides encoding BY24.5L with a double expression cassette; or vice versa. The recombinant vectors were sequenced and verified to be correct then used for expression. The expressed anti-VEGFR antibody-CD80 fusion protein was named fusion protein BY24.5.

(5) Synthesis of Nucleotides Encoding Fusion Protein Containing CD80 Extracellular Domain, Immunoglobulin Fc Region and VEGFR Extracellular Receptor Functional Domain and Construction of Expression Vectors

According to the sequence of the CD80 extracellular domain in Table 1, the IgG4Fc sequence in Table 6, and the VEGFR functional region sequence in Table 5 (VEGFR1-D2/VEGFR2-D3) were optimized to be suitable for expression in Chinese hamster ovarian cancer cells (CHO). And Shanghai Jierui Bioengineering Co., Ltd. was entrusted to synthesize the following polynucleotide sequence shown in SEQ ID NO: 87. The CD80-Fc-VEGFR fusion protein produced after the expression of the nucleotide sequence is also referred to herein as the fusion protein BY24.12.

Nucleotide sequence of fusion protein BY24.12 (CD80-Fc-VEGFR) (SEQ ID NO: 87)

CTCGAGGCCACCGCCACCATGGAGACCGACACACTCCTCCTGTGGGTGCT
GCTGCTGTGGGTGCCTGGCTCCACTGGCGTCATCCATGTGACCAAAGAGG
TCAAAGAAGTCGCAACTCTGTCTTGCGGACACAACGTCTCCGTCGAAGAA
CTCGCCCAGACTAGAATATATTGGCAGAAGGAAAAGAAGATGGTGCTCAC
CATGATGTCCGGCGATATGAACATTTGGCCTGAGTACAAGAACCGGACAA
TCTTCGATATTACTAATAACCTGAGTATCGTGATTCTCGCTCTGCGCCCT
AGCGACGAGGGCACATACGAGTGTGTGGTGCTGAAGTACGAGAAGGATGC
ATTCAAGCGGGAGCACCTCGCAGAGGTGACACTCTCCGTGAAGGCCGACT
TCCCAACCCCATCTGACAAGCGCGTGGAGTCCAAGTACGGCCCTCCTTGC
CCTCCTTGCCCTGCCCCTGAGTTCCTGGGCGGCCCTTCCGTGTTCCTGTT
CCCTCCTAAGCCTAAGGACACCCTGATGATCTCCCGCACCCCTGAGGTGA
CCTGCGTGGTGGTGGACGTGTCCCAGGAGGACCCTGAGGTGCAGTTCAAC
TGGTACGTGGACGGCGTGGAGGTGCACAACGCCAAGACCAAGCCTCGCGA
GGAGCAGTTCAACTCCACCTACCGCGTGGTGTCCGTGCTGACCGTGCTGC
ACCAGGACTGGCTGAACGGCAAGGAGTACAAGTGCAAGGTGTCCAACAAG
GGCCTGCCTTCCTCCATCGAGAAGACCATCTCCAAGGCCAAGGGCCAGCC
TCGCGAGCCTCAGGTGTACACCCTGCCTCCTTCCCAGGAGGAGATGACCA
AGAACCAGGTGTCCCTGACCTGCCTGGTGAAGGGCTTCTACCCTTCCGAC
ATCGCCGTGGAGTGGGAGTCCAACGGCCAGCCTGAGAACAACTACAAGAC
CACCCCTCCTGTGCTGGACTCCGACGGCTCCTTCTTCCTGTACTCCCGCC
TGACCGTGGACAAGTCCCGCTGGCAGGAGGGCAACGTGTTCTCCTGCTCC
GTGATGCACGAGGCCCTGCACAACCACTACACCCAGAAGTCCCTGTCCCT
GTCCCTGGGCGGCGGAGGATCTGGCGGCGGAGGCAGTGGAGGCGGCGGAA
GTGCTTCCGACACCGGCCGCCCTTTCGTGGAGATGTACTCCGAGATCCCT
GAGATCATCCACATGACCGAGGGCCGCGAGCTGGTGATCCCTTGCCGCGT
GACCTCCCCTAACATCACCGTGACCCTGAAGAAGTTCCCTCTGGACACCC
TGATCCCTGACGGCAAGCGCATCATCTGGGACTCCCGCAAGGGCTTCATC
ATCTCCAACGCCACCTACAAGGAGATCGGCCTGCTGACCTGCGAGGCCAC
CGTGAACGGCCACCTGTACAAGACCAACTACCTGACCCACCGCCAGACCA
ACACCATCATCGACGTGGTGCTGTCCCCTTCCCACGGCATCGAGCTGTCC
GTGGGCGAGAAGCTGGTGCTGAACTGCACCGCCCGCACCGAGCTGAACGT
GGGCATCGACTTCAACTGGGAGTACCCTTCCTCCAAGCACCAGCACAAGA
AGCTGGTGAACCGCGACCTGAAGACCCAGTCCGGCTCCGAGATGAAGAAG
TTCCTGTCCACCCTGACCATCGACGGCGTGACCCGCTCCGACCAGGGCCT
GTACACCTGCGCCGCCTCCTCCGGCCTGATGACCAAGAAGAACTCCACCT
TCGTGCGCGTGCACGAGTAAGAATTC

The amino acid sequence of the fusion protein BY24.12 (CD80-Fc-VEGFR) (SEQ ID NO: 88)

METDTLLLWVLLLWVPGSTGVIHVTKEVKEVATLSCGHNVSVEELAQTRI
YWQKEKKMVLTMMSGDMNIWPEYKNRTIFDITNNLSIVILALRPSDEGTY
ECVVLKYEKDAFKREHLAEVTLSVKADFPTPSDKRVESKYGPPCPPCPAP
EFLGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEAL
HNHYTQKSLSLSLGGGGSGGGGSGGGGSASDTGRPFVEMYSEIPEIIHMT
EGRELVIPCRVTSPNITVTLKKFPLDTLIPDGKRIIWDSRKGFIISNATY
KEIGLLTCEATVNGHLYKTNYLTHRQTNTIIDVVLSPSHGIELSVGEKLV
LNCTARTELNVGIDFNWEYPSSKHQHKKLVNRDLKTQSGSEMKKFLSTLT
IDGVTRSDQGLYTCAASSGLMTKKNSTFVRVHE

Using the same method as in Example 1(1) above, the nucleotides encoding BY24.12 digested by XhoI-EcoRI double enzymes were linked to glutamine synthetase high-efficiency expression vectors with double expression cassettes (Patent No.: CN104195173B, obtained from Beijing Biyang Biotechnology Co., Ltd.). The recombinant vectors were sequenced and verified to be correct then used for expression. The expressed fusion protein was named fusion protein BY24.12.

### Example 2. Expression and Purification of Target Protein

(1) Transient Expression of Target Protein

293 F (available from Invitrogen Inc., catalog No.: 11625-019) was suspended in serum-free CD293 broth (available from Invitrogen Inc. catalog No.: 11913-019). The cell culture was centrifuged prior to transfection, cell precipitation was obtained, cells were suspended with fresh serum-free CD293 broth, and the cell concentration was adjusted to 1×10⁶ cells/mL. The cell suspension was placed in a flask. Taking 100 ml cell suspension as an example, 250 ug of each recombinant expression vector plasmid DNA containing the target gene prepared in Example 1 and 500 ug of polyethylenimine (PEI) (Sigma, catalog number: 408727) were added separately and mixed uniformly in 1 ml serum-free CD 293 culture medium, after standing at room temperature for 8 minutes, the PEI/DNA suspension was added dropwise to a shake flask containing 100 mL of the cell suspension. Gently mixed uniformly, and placed in a 5% CO₂, 37° C. shaker (120 revolutions per minute). The culture supernatant was collected after 5 days.

(2) Purification of Expressed Protein

The protein of interest present in the culture supernatant collected by Example 2 (1) was purified by HiTrap MabSelect SuRe 1 ml column (GE Healthcare Life Sciences Product, Catalog No. 11-0034-93) equilibrated with a pH 7.4 PBS solution. Briefly, a PBS solution of pH 7.4 was used to equilibrate HiTrap MabSelect SuRe 1 ml column at 10 column bed volumes, and the flow rate was 0.5 mL/min. After the culture supernatant collected in Example 2 (1) was filtered with a 0.45 μm filter membrane, the sample was loaded to the HiTrap MabSelect SuRe 1 ml column equilibrated a PBS solution of pH 7.4;

After supernatant was loaded, the column was first washed with PBS solution of pH 7.4 at a flow rate of 0.5 mL/min for 5-10 column bed volumes, and then eluted with 100 mM citric acid buffer (pH 4.0) at a flow rate of 0.5 mL/min. The elution peak was collected, and the proteins of interest BY18.1, BY31.19, BY24.4, BY24.5, BY24.12, respectively, were present in the elution peaks.

The purity and molecular weight of the proteins of interest BY18.1, BY31.19, BY24.4, BY24.5, BY24.5, BY24.12 respectively, were analyzed by SDS-PAGE electrophoresis and stained with Coomassie blue in the presence of a reducing agent (5 mM 1,4-dithiothreitol). The results are shown in Table 8 below, where theoretical predicted values and the actual measured values of the molecular weight are given. Since there is a glycosylation effect on the protein in the eukaryotic expression system, the actual measured value of the molecular weight is slightly higher than the theoretical predicted value.

TABLE 8
The molecular weight of the purified expressed protein

| | | The size of theoretical | The size of actual |
|---|---|---|---|
| | | predicted molecular | molecular |
| Name of protein | Subunit | weight(kDa) | weight(kDa) |
| Fusion protein | First | 23 | 27 |
| BY24.4 | subunit | | |
| | Second | 62 | 70 |
| | subunit | | |
| Fusion protein | First | 23 | 27 |
| BY24.5 | subunit | | |
| | Second | 74 | 70 |
| | subunit | | |
| Fusion protein | Any | 63 | 72 |
| BY24.12 | subunit | | |
| Fusion protein | Any | 40 | 45 |
| BY31.19 | subunit | | |
| Antibody | Light | 23 | 27 |
| BY18.1 | chain | | |
| | Heavy | 48 | 50 |
| | chain | | |

### Example 3. Detection of Specific Binding Effects Using ELISA Methods

Recombinant human CD28 (the product of Beijing Yiqiao Shenzhou Biotechnology Co., Ltd., catalog No.: 50103-MO8H), recombinant human PD-L1 (Beijing Biosciences Biotechnology Co., Ltd., catalog No. PD1-H5229) and recombinant human CTLA-4 (the product of Beijing Yiqiao Shenzhou Biotechnology Co., Ltd., catalog No.: 11159-H08H) was diluted to 0.5m/ml, 0.25m/ml and 1.0m/ml and coated with 96-well ELISA plate (Corning company, catalog No.: 42592). The fusion proteins BY24.4, BY24.5, BY24.12, and BY31.19 purified in the above-mentioned Example 2(2) were diluted to 2000m/ml, and then a 3-fold serial dilution was carried out and was diluted to 16 gradients totally, each concentration gradient is tested by multiple holes. 50 μl of the diluted sample was added to the 96-well plate coated with recombinant human CD28, recombinant human CTLA-4 or recombinant human PD-L1, and incubated at 37° C. for 2 hours. After washing 3 times, goat anti-human secondary antibody labeled with horseradish peroxidase (product of Beijing Zhongshan Jinqiao Company, product No.: ZDR-5301) was added, and incubated at 37° C. for 1 hour. After washing 3 times, 3,3′,5,5′-tetramethylbenzidine (TMB) substrate color developing solution (Beijing Kangwei Century Biotechnology Co., Ltd., product No.: CW0050) was added 50 μl/well. 10 minutes later, 2N H₂SO₄ was added to stop the color development. The absorbance OD value of each well at 450 nm was measured using an ELISA reader.

The protein concentration of fusion proteins BY24.4, BY24.5, BY24.12, and BY31.19 against the absorbance OD value was plotted using GraphPadrism5 software, and the data was fitted to produce a half maximum effective concentration EC50 value of the specific binding effect mediated by fusion protein. The results are shown in Table 9 below.

TABLE 9
The binding effect of each fusion protein on human
PD-L1, human CD28 and human CTLA-4

| | Binding of | Binding of | Binding of |
|---|---|---|---|
| | hPD-L1 | hCD28 | hCTLA-4 |
| Name of protein | EC50 (μg/ml) | EC50 (μg/ml) | EC50 (μg/ml) |
| Fusion protein | 6.352 | 23.05 | 2.270 |
| BY24.4 | | | |
| Fusion protein | 5.167 | 35.07 | 3.751 |
| BY24.5 | | | |
| Fusion protein | 3.748 | 13.16 | 2.890 |
| BY24.12 | | | |
| Fusion protein | 2.20 | 24.33 | 1.924 |
| BY31.19 | | | |

The ELISA results showed that the multi-targeting fusion protein BY24.4, BY24.5, BY24.12, and BY 31.19 as a positive control can bind to recombinant human PD-L1, recombinant human CD28 and CTLA-4. Each fusion protein had the strongest binding ability to CTLA-4, followed by PD-L1, and the weakest binding to CD28.

Example 4 the affinity of the protein of interest to the target in the present invention was determined by Biacore T100.

The surface plasmon resonance measurement was performed on T 100 Instruments (GE Healthcare Biosciences, AB, Sweden) at 25° C.

First, the anti-IgG antibody (GE Healthcare Life Sciences, catalog No.: BR-1008-39) was covalently immobilized on the CM5 chip by amide coupling. The CM5 chip was activated using 60 μl of N-ethyl-N′-(3-dimethylaminopropyl) carbodiimide hydrochloride (EDC) and 60 μl of N-hydroxysuccinimide (NHS), and 95 μl dilution buffer HBST (0.1M HEPES, 1.5M NaCl, pH7.4, and 0.005% Tween 20) was added to 5 μl anti-IgG antibody and filtered through 0.2 um filter membrane, then anti-IgG antibody was covalently immobilized on the CM5 chip by amide coupling, yielding a capture system of about 9000 to 14,000 resonant units (RU). 120 μl ethanolamine was used to block the CM5 chip.

Then, each protein of interest in the present invention prepared in Example 2 was diluted to 5 μg/ml, and the diluent was injected at a flow rate of 10 μL/min for 2 minutes, each protein of interest in the present invention prepared in Example 2 was covalently captured on the surface of the CM5 chip by their respective Fc domains. The resulting composite was stabilized by cross-linking with EDC/NHS to avoid baseline drift during measurement and regeneration.

The binding antigen PD-1 (the product of Beijing Yiqiao Shenzhou Biotechnology Co., Ltd., catalog No.: 10377-H08H), VEGF165 (the product of Beijing Yiqiao Shenzhou Biotechnology Co., Ltd., catalog No.: 11066-HNAH), and mouse VEGF164 (the product of Beijing Yiqiao Shenzhou Biotechnology Co., Ltd., catalog number: 50159-MNAB) and VEGFR2 (the product of Beijing Yiqiao Shenzhou Biotechnology Co., Ltd., catalog No.: 10012-H02H1) are formulated into the following concentration gradients: 7 nM, 22 nm, 66 nM, 200 nM, 600 nM. The binding was measured by injecting each concentration at a flow rate of 30 μl/min for 180 seconds and a dissociation time of 600 seconds. The surface was regenerated by washing with a 3M MgCl2 solution at a flow rate of 10 μL/min for 30 seconds. Data analysis BIA was performed by evaluation software (BIA evaluation 4.1 software, from GE Healthcare Biosciences AB, Sweden), and the affinity data shown in Table 10 below were obtained.

TABLE 10
The binding of each protein of interest to the target

| Name of | | | | |
|---|---|---|---|---|
| protein of interest | Target | ka(1/Ms) | kd(1/s) | KD(M) |
| Fusion protein | VEGF-A | 2.221E+6 | 3.407E−5 | 1.534E−11 |
| BY24.4 | | | | |
| Fusion protein | VEGFR2 | 6.82E+5 | 5.57E−5 | 8.17E−11 |
| BY24.5 | | | | |
| Fusion protein | human- | 3.15E+6 | 1.25E−4 | 3.98E−11 |
| BY24.12 | VEGF-A | | | |
| Fusion protein | mouse | 9.18E+5 | 3.13E−5 | 3.41E−11 |
| BY24.12 | VEGF-A | | | |
| Antibody BY18.1 | PD-1 | 6.66E+5 | 1.58E−3 | 2.37E−9 |

The KD(M) of the fusion proteins BY24.4, BY24.5, BY24.12 and their respective targets are all lower than 10⁻⁹M, indicating that they have high affinity with their respective targets, especially the fusion protein BY24.12 can bind to both mice and human VEGF-A with high affinity and the difference is not very big.

### Example 5. The Effect of the Protein of Interest in the Present Invention and its Composition on the Secretion of IFN-γ in the Mixed Lymphocyte Reaction (MLR)

CD4+T lymphocytes and dendritic cells (DC) were available from Beijing Shihe Biotechnology Co., Ltd., and the CD4+T lymphocytes and dendritic cells (DC) were derived from different healthy people. CD4+T lymphocytes and dendritic cells (DC) were seeded in 96-well cell culture plates at 1×10⁵ cells/well and 1×10⁴ cells/well, respectively. The experiments were divided into 7 groups, namely BY18.1 group (1.0 μg/ml), BY24.4 group (1.14 μg/ml), BY24.4 (1.14 μg/ml)+BY18.1 (1.0 μg/ml) group, BY24.5 (1.14 μg/ml) group, BY24.5 (1.14 μg/ml)+BY18.1 (1.0 μg/ml) group, BY24.12 (0.84 μg/ml) group and BY24.12 (0.84 μg/ml)+BY18.1 (1.0 μg/ml) group. 3 replicate wells in each group, antibody BY18.1, fusion protein BY24.4, BY24.5, BY24.12, or antibody BY18.1 and each combination of BY24.4, BY24.5, BY24.12 were added as required to the final concentration shown. Finally, 1640 medium containing 10% fetal bovine serum was added and the final volume was 200p1. Cultured at 37 C, 5% CO₂.

After 5 days of culture, there were many clump cells in all the seven experimental groups, and there was a significant portion of the presence of shuttle and adherent cells. The IFN-γ expression level of each group was detected by an IFN-γ kit (Cargo No.: EH 008-96 ELISA).

The detection results were as follows: Compared with the antibody BY18.1 group (2221.8±364.5 pg/ml), the secretion of IFN-γ in the fusion protein BY24.4 group alone (924.1±221.9 pg/ml), BY24.5 group (760.1±286.8 pg/ml) and BY24.12 group (793.4±139.2 pg/ml) was all significantly lower than that of the antibody BY18.1 group (P<0.01); but after the fusion proteins BY24.4, BY24.5 and BY24.12 were added to BY18.1 respectively, the secretion of IFN-γ all increased significantly. The secretion of IFN-γ in each group was respectively as follows: BY24.4+BY18.1 group (3494.2±364.5 pg/ml), BY24.5+BY18.1 group (3523.8±465.1 pg/ml) and BY24.12+BY18.1 group (3801.8±702.2 pg/ml). It can be seen that the combination of fusion proteins BY24.4, BY24.5 and BY24.12 respectively with BY18.1 can lead to a significant increase in the secretion of IFN-γ (P<0.01), and it indicated that the antibody BY18.1 (PD-1 antibody) in combination with the multi-targeting fusion protein BY24.4, BY24.5 and BY24.12 of the present invention has a significant synergistic effect on the secretion of IFN-γ.

### Example 6. The Synergistic Effect of the Multi-Targeting Fusion Protein of the Present Invention and PD-1 Antibody

The main purpose of this example is to explore the synergistic effect of the multi-targeting fusion protein and the PD-1 antibody in anti-tumor in vivo in the present invention, therefore, the doses of the PD-1 antibody and each fusion protein are all low doses because high dose may have a better tumor suppressive effect and no synergy between the PD-1 antibody and each fusion protein can be observed.

Mouse-derived colon cancer cells CT26 (ATCC) in RPMI-1640 medium were seeded in rib subcutaneous at right front of about 18 g, 6 weeks old female BALB/c mouse, 100 μl/mouse, the amount of inoculation is 1×10⁶ cells/mouse. The time of CT26 inoculation of mouse-derived colon cancer cells was set to day 0. When the average tumor volume reached about 93 mm³, the tumor-bearing mice were randomly grouped, 6 mice per group, and 5 groups in total. The molar amounts were calculated according to the size of the molecular weight, the fusion protein BY 31.19, BY 24.12, and the anti-MPD-1 antibody were administered in equivalent molar amounts. Specifically, the grouping and dosage were as follows: vehicle (PBS) control group; fusion protein BY31.19 (1.6 mg/kg) group; fusion protein BY24.12 (2.5 mg/kg) group; anti-mPD-1 (3.0 mg/kg, available from BioXcell, clone No.: RMP1-14, product No.: BE0146) group; fusion protein BY24.12 (2.5 mg/kg)+anti-mPD-1 (3 mg/kg) group. Dosing twice a week. The tumor volume was measured three times a week. While the tumor volume was being measured, the mice were weighed, and the relationship between the changes in the mouse body weight and tumor volume and the administration time was recorded. At the end of the experiment, the mice were euthanized, serum and tumors were collected, and the serum was cryopreserved at −80° C. After the tumors were weighed and photographed, they were formalin-fixed and paraffin-embedded (FFPE) to prepare tissue samples for later use. The data was analyzed statistically and the relative tumor volume ratio (T/C) % (That is: (the mean value of tumor volume change in the drug-administered group/the mean value of tumor volume change in the PBS vehicle control group)×100%) and tumor growth inhibition rate (Tumor Growth Inhibition %) of each treatment group and vehicle control group were calculated, which was (1-T/C) %.

At the end of the experiment, the tumor growth inhibition rates (TGI %) of BY31.19 group, BY24.12 group, PD-1 group, BY24.12+anti-mPD-1 group were 15%, 17%, 24%, and 47% (calculated by tumor volume), all have different degrees of inhibition. The inhibitory effect of the immune fusion protein BY24.12 and PD-1 antibody in combination was significantly better than that of BY24.12 and PD-1 alone, so there is a synergistic effect between the two.

The animals in each group continued to increase their body weight after the administration, and the overall condition was good, no obvious abnormality, and there was no significant drug withdrawal or death in each group.

1. A multi-targeting fusion protein that blocks the growth of vascular endothelial cells and activates T cells, which comprises (i) a vascular endothelial growth inhibitor domain; (ii) an immunoglobulin Fc domain; and (iii) CD80 extracellular domain (ECD).

2. The multi-targeting fusion protein according to claim 1, wherein the (i) comprises an antigen-binding fragment derived from an anti-VEGF antibody and/or an anti-VEGFR antibody and/or a VEGFR extracellular receptor functional domain;
preferably, the antigen-binding fragment of the anti-VEGF antibody and/or the anti-VEGFR antibody is the Fab, Fab′, F(ab′)2, Fv, single-chain Fv of the anti-VEGF antibody and/or the anti-VEGFR antibody; more preferably, the antigen-binding fragment of the anti-VEGF antibody and/or the anti-VEGFR antibody comprises all six heavy chain CDRs and light chain CDRs that are contained in paired heavy chain variable region sequence/light chain variable region sequence selected from the group consisting of SEQ ID NO: 1/2, 3/4, and 5/6, or a sequence having one, two, three, four, or five amino acid changes (e.g., amino acid replacement or deletion) from one or more CDR(s) in all the six heavy chain CDRs and light chain CDRs; further preferably, the antigen-binding fragment of the anti-VEGF antibody and/or the anti-VEGFR antibody comprises the paired heavy chain variable region sequence/light chain variable region sequence selected from the group consisting of SEQ ID NO: 1/2, 3/4 and 5/6, or a sequence having at least 90%, 91%, 92%, 93%, 94%, 95%, 96%, 97%, 98%, 99% or more sequence identity to the paired heavy chain variable region sequence/light chain variable region sequence; most preferably, the antigen-binding fragment of the anti-VEGF antibody and/or the anti-VEGFR antibody is the Fab of Bevacizumab, Ranibizumab or Ramucirumab;
preferably, the VEGFR extracellular receptor functional domain comprises the immunoglobulin-like domain 2 of VEGFR1 and the immunoglobulin-like domain 3 of VEGFR2; or the VEGFR extracellular receptor functional domain comprises the immunoglobulin-like domain 2 of VEGFR1 and the immunoglobulin-like domain 3 of VEGFR2 and the immunoglobulin-like domain 4 of VEGFR2; or the VEGFR extracellular receptor functional domain comprises the immunoglobulin-like domain 2 of VEGFR1; more preferably, the VEGFR extracellular receptor functional domain has any amino acid sequence selected from SEQ ID NO: 7-9 or an amino acid sequence having at least 90%, 91%, 92%, 93%, 94%, 95%, 96%, 97%, 98%, 99% or more sequence identity to the amino acid sequence shown in SEQ ID NO: 7-9.

3. The multi-targeting fusion protein according to claim 1, wherein the (ii) is a human immunoglobulin Fc domain; preferably, the (ii) is a human IgG1, IgG2, IgG3 or IgG4 Fc domain; more preferably, the (ii) comprises the Fc domain of the amino acid sequence shown in SEQ ID NO: 10, 11 or 12, or comprises the Fc domain having at least 90%, 91%, 92%, 93%, 94%, 95%, 96%, 97%, 98%, 99% or more sequence identity to the amino acid sequence shown in SEQ ID NO: 10, 11 or 12.

4. The multi-targeting fusion protein according to claim 1, wherein the (iii) comprises human CD80 ECD; preferably, the (iii) comprises human CD80 IgV or human CD80 IgVIgC; preferably, the (iii) has the amino acid sequence shown in SEQ ID NO: 13 or 14 or an amino acid sequence having at least 90%, 91%, 92%, 93%, 94%, 95%, 96%, 97%, 98%, 99% or more sequence identity to the amino acid sequence shown in SEQ ID NO: 13 or 14.

5. The multi-targeting fusion protein according to claim 1, which further comprises a peptide linker between (i), (ii) and/or (iii); preferably, the peptide linker comprises one or more amino acids, more preferably at least 5 amino acids, and most preferably a peptide linker selected from SEQ ID NO: 20-46.

6. The multi-targeting fusion protein according to claim 1, wherein the fusion protein is effectively connected in the order of (i), (ii) and (iii); the order of (iii), (i) and (ii); or the order of (iii), (ii) and (i) from N-terminal to C-terminal.

7. The multi-targeting fusion protein according to claim 6, which comprises
(a) A full-length anti-VEGF antibody, a full-length anti-VEGFR antibody, or a full-length anti-VEGF and VEGFR bispecific antibody; and one CD80 ECD effectively connected to the C-terminal of each of the two heavy chains of the antibody;
(b) A full-length anti-VEGF antibody, a full-length anti-VEGFR antibody, or a full-length anti-VEGF and VEGFR bispecific antibody; and one CD80 ECD effectively connected to the N-terminal of each of the two heavy chains of the antibody; and one CD80 ECD effectively connected to the N-terminal of each of the two light chains of the antibody;
(c) A CD80 ECD; an immunoglobulin Fc domain in dimer form effectively connected at the C-terminal of a CD80 ECD; and an antigen-binding fragment derived from an anti-VEGF antibody and/or an anti-VEGFR antibody effectively connected at the C-terminal of an immunoglobulin Fc domain in dimer form; or
(d) A CD80 ECD; an immunoglobulin Fc domain in dimer form effectively connected at the C-terminal of a CD80 ECD; and a VEGFR extracellular receptor functional domain effectively connected to the C-terminal of the immunoglobulin Fc domain in dimer form;
Preferably, the antibody is an IgG class antibody, particularly an IgG₁ subclass, an IgG₂ subclass, an IgG₄ subclass antibody; and more particularly an IgG₄ subclass antibody; also preferably, the IgG₄ subclass antibody contains amino acid replacement at the 5228 position of the Fc domain, more preferably amino acid replacement S228P; further preferably, the light chain type of the antibody is κ type or λ type, preferably κ type;
Preferably, the full-length anti-VEGF antibody is Bevacizumab, and the full-length anti-VEGFR antibody is Ramucirumab.

8. The multi-targeting fusion protein according to claim 1, which is selected from
(1) A fusion protein comprising the first subunit of the fusion protein of SEQ ID NO: 80 and the second subunit of the fusion protein of SEQ ID NO: 82;
(2) A fusion protein comprising the first subunit of the fusion protein of SEQ ID NO: 84 and the second subunit of the fusion protein of SEQ ID NO: 86;
(3) A fusion protein comprising the fusion protein subunit of SEQ ID NO: 88.

9. A polynucleotide encoding the multi-targeting fusion protein of claim 1.

10. A vector, preferably an expression vector, most preferably a glutamine synthetase expression vector with double expression cassettes, the vector comprises the polynucleotide of claim 9.

11. A host cell, which comprises the polynucleotide of claim 9 or a vector, preferably the host cell is a CHO, HEK293 or NSO cell;
wherein the vector comprises the polynucleotide of claim 9, preferably the vector is an expression vector, more preferably, the vector is a glutamine synthetase expression vector with double expression cassettes.

12. A method for producing the multi-targeting fusion protein of claim 1, which comprises step (i) culturing a host cell under conditions suitable for expressing the multi-targeting fusion protein, and step (ii) recovering the fusion protein;
wherein
the host cell comprises a polynucleotide, wherein the polynucleotide encoding the multi-targeting fusion protein of claim 1; or
the host cell comprises a vector, wherein the vector comprises a polynucleotide, wherein the polynucleotide encoding the multi-targeting fusion protein of claim 1, preferably the vector is an expression vector, more preferably, the vector is a glutamine synthetase expression vector with double expression cassettes;
preferably the host cell is a CHO, HEK293 or NSO cell.

13. A pharmaceutical composition, which comprises the multi-targeting fusion protein according to claim 1 and an anti-PD-1 antibody, preferably, the anti-PD-1 antibody comprises all six heavy chain CDRs and light chain CDRs contained in the paired heavy chain variable region sequence/light chain variable region sequence selected from the group consisting of SEQ ID NO: 47/48, 49/50, 51/52, 53/54, 55/56, 57/58, 59/60, 61/62, 63/64, 65/66, 67/68, 69/70 and 71/72, or a sequence having one, two, three, four, or five amino acid changes (e.g., amino acid replacement or deletion) from one or more CDR(s) in all six heavy chain CDRs and light chain CDRs; further preferably, the anti-PD-1 antibody comprises paired heavy chain variable region sequence/light chain variable region sequence selected from SEQ ID NO: 47/48, 49/50, 51/52, 53/54, 55/56, 57/58, 59/60, 61/62, 63/64, 65/66, 67/68, 69/70 and 71/72, or a sequence having at least 90%, 91%, 92%, 93%, 94%, 95%, 96%, 97%, 98%, 99% or more sequence identity to the paired heavy chain variable region sequence/light chain variable region sequence; most preferably, the anti-PD-1 antibody is selected from Nivolumab, Pidilizumab and Pembrolizumab.

14. A method for treating or preventing a cancerous disease (e.g., a solid tumor or a soft tissue tumor) in an individual, comprising a step of administering to the individual with an effective amount of the multi-targeting fusion protein according to claim 1; preferably the cancerous disease is melanoma, breast cancer, colon cancer, esophageal cancer, gastrointestinal stromal tumor (GIST), kidney cancer (e.g., renal cell carcinoma), liver cancer, non-small cell lung cancer (NSCLC), ovarian cancer, pancreatic cancer, prostate cancer, head and neck tumors, gastric cancer, or a hematological malignancy (e.g., lymphoma); in particular, the disease is colon cancer or triple negative breast cancer; preferably, wherein the individual is a mammal, more preferably a human.

15. A method for treating or preventing a cancerous disease (e.g., a solid tumor or a soft tissue tumor) in an individual, comprising a step of administering to the individual with an effective amount of the pharmaceutical composition according to claim 13; preferably the cancerous disease is melanoma, breast cancer, colon cancer, esophageal cancer, gastrointestinal stromal tumor (GIST), kidney cancer (e.g., renal cell carcinoma), liver cancer, non-small cell lung cancer (NSCLC), ovarian cancer, pancreatic cancer, prostate cancer, head and neck tumors, gastric cancer, or a hematological malignancy (e.g., lymphoma); in particular, the disease is colon cancer or triple negative breast cancer; preferably, wherein the individual is a mammal, more preferably a human.
